(19) United States
(12) Patent Application Publication
(10) Pub. No.: US 2012/0015400 A1
(43) Pub. Date: Jan. 19, 2012

(54) HCL-K1 Polypeptide Which Offers Collectin Activity

(75) Inventors: Nobutaka Wakamiya, Hokkaido (JP); Katsuki Otani, Hokkaido (JP); Takashi Sakamoto, Nara (JP); Yuichiro Kishi, Wakayama (JP); Hiroyuki Keshi, Osaka (JP); Aya Keshi, Osaka (JP)

(73) Assignees: ASAHIKAWA MEDICAL COLLEGE, Hokkaido (JP); FUSO PHARMACEUTICAL INDUSTRIES, LTD., Osaka (JP)

(21) Appl. No.: 13/087,989

(22) Filed: Apr. 15, 2011

Related U.S. Application Data
(63) Continuation of application No. 12/296,085, filed on Jul. 22, 2009, now abandoned.
Application No. 12/296,085 is a continuation-in-part of application No. 10/258,105, filed on Mar. 19, 2003, now abandoned.

(30) Foreign Application Priority Data
Apr. 21, 2000 (JP) 2000-120358
Apr. 5, 2006 (JP) 2006-104667

(51) Int. Cl. C12P 21/00; C12N 15/63

(52) U.S. Cl. 435/69.1; 435/320.1

(57) ABSTRACT

Disclosed is the novel hCL-K1 polypeptide which offers collectin activity. This polypeptide consists of consecutive 271 amino acids set out in SEQ ID NO: 2 and does not bind to both maltose and N-acetylgalactosamine.

### TECHNICAL FIELD

The present invention relates to purified and isolated human polypeptide which offers collectin activity.

### BACKGROUND ART

Complement systems that play an important role in a body defense mechanism are known that it includes a classic pathway in which an immunoglobulin serves as a recognition molecule followed by activation of C1 that is the first component of the complement, and an alternative pathway wherein C3, which is the third component of the complement, is directly coupled to foreign substances such as bacteria. In addition to these pathways of the complement activation, a lectin pathway was illustrated wherein a mannose binding protein (hereinafter referred to as ‘MBP’), which is a serum lectin, activates the complement system through the direct recognition of and coupling with a carbohydrate chain on the surface of the foreign substance, in recent years [Non-Patent Publication 1].

MBP is a C type lectin which specifically binds to mannose, N-acetylglucosamine and so on in the presence of calcium, their structure comprises a collagen-like domain containing at least one amino acid sequence of (Gly-Xaa-Yaa)ₙ, and carbohydrate chain recognition domain (CRD). Lectins having a collagen-like domain and carbohydrate chain recognition domain are generically called as collectin [Non-Patent Publication 2], which include collectin-43 (CL-43), surfactant protein A (SP-A), surfactant protein D (SP-D), bovine conglutinin (BKg) and the like, in addition to MBP.

Collectin has an opsonic activity, which is believed to participate in fundamental immunity against a variety of microorganisms such as bacteria and viruses [Non-Patent Publications 3-6]. With reference to FIG. 1, these collectins A are known to be constituted from a basic structure containing characteristic domains such as carbohydrate chain recognition domain B and collagen-like domain C [Non-Patent Publication 7]. This basic structure forms a subunit through composing a triple helix at the collagen-like domain C, and thus these subunits further form an oligomer structure such as trimer, tetramer, and hexamer.

Recently, collectins were suggested to participate in non-specific immune response, e.g., it was reported that for example, they are playing important roles in neutralizing and excluding various microorganisms in infants having insufficient maternal antibodies from mother or having specific defense systems which were insufficiently developed [Non-Patent Publication 8]. Moreover, results of investigation are reported involving in roles of these collectins in the body defense system of a host, which for example, suggest that the host becomes more susceptible to infections through the lowered concentration of MBP in blood resulting from genetic mutation of MBP [Non-Patent Publication 9]. In addition, it was reported that serum MBP content shows a lowered level upon the failure of opsonization [Non-Patent Publication 10], whilst bacterial infections readily occur [Non-Patent Publication 11]. Therefore, MBP can be believed to play important roles in an immune system.

The present inventors previously found that BKg and MBP inhibit infections by H1 and H3 types influenzae A viruses as well as a haemagglutination activity [Non-Patent Publications 12-13].

Thereafter, a cDNA clone encoding BKg was obtained, and the relevance between BKg and SP-D and the like has been also found [Non-Patent Publication 14].
Non-Patent Publication 1: Sato, T. et al., Int. Immunol., 6, pp. 665-669 (1994)
Non-Patent Publication 2: Malhotora, R. et al., Eur. J. Immunol., 22, pp. 1437-1445 (1992)
Non-Patent Publication 3: Kawasaki, N. et al., J. Biochem., 106, pp. 483-489 (1989)
Non-Patent Publication 4: Ikeda, K. et al., J. Biol. Chem., 262, pp. 7451-7454 (1987)
Non-Patent Publication 5: Ohta, M. et al., J. Biol. Chem., 265, pp. 1980-1984 (1990)
Non-Patent Publication 6: Summerfield, J. A. et al., Lancet, 345, p. 886 (1995)
Non-Patent Publication 7: Malhortra et al., Eur. J. Immunol., 22, pp. 1437-1445 (1992)
Non-Patent Publication 8: Super et al., Lancet, 2, pp. 1236-1239 (1989)
Non-Patent Publication 9: Sumiya et al., Lancet, 337, pp. 1569-1570 (1991)
Non-Patent Publication 10: Madsen, H. O. et al., Immuno genetics, 40, pp. 37-44 (1994)
Non-Patent Publication 11: Garred, P. et al., Lancet, 346, pp. 941-943 (1995)
Non-Patent Publication 12: Wakamiya et al., Glycoconjugate J., 8, p. 235 (1991)
Non-Patent Publication 13: Wakamiya et al., Biochem. Biophys. Res. Comm., 187, pp. 1270-1278 (1992)
Non-Patent Publication 14: Suzuki et al., Biochem. Biophys. Res. Comm., 191, pp. 335-342 (1993)

### DISCLOSURE OF THE INVENTION

### Problems to be Solved by the Invention

As stated above, collectins are substances to which usefulness in the elucidation of body defense mechanisms and utilities as a biologically active substance are expected. In particular, the finding of novel molecular species belonging to this family may greatly contribute in various medical fields and biological fields in addition to the therapy of infectious diseases.

Accordingly, the object of the present invention is to provide novel polypeptides that offer immunological activity (hereinafter referred to as ‘collectin activity’) which systematically involves with fundamental human immunity by, for example, activating complement systems.

### Means to Solve the Problems

In consideration of the problems known in the art as stated previously, the present inventors performed research continuously on any novel polypeptide, in particular, those taken from human species and offer industrially available collectin activity, and have realized the present invention.

Namely, the merit of the present inventions are;

(1) Purified and isolated polypeptide which consists of consecutive 271 amino acids set out in SEQ ID NO: 2 and does not bind to both maltose and N-acetylgalactosamine;

(2) Purified and isolated polypeptide which consists of consecutive 223 amino acids set out in SEQ ID NO: 5;

(3) Purified and isolated polypeptide which consists of consecutive 247 amino acids set out in SEQ ID NO: 8; and

(4) Purified and isolated polypeptide which consists of consecutive 247 amino acids set out in SEQ ID NO: 11.

The other embodiment of the present invention also provides the polypeptides wherein one to several amino acids in the foregoing polypeptides is deleted, substituted or added, and offer collectin activity substantially similar to those realized by the foregoing polypeptides.

Then the other embodiment of the present invention provides the polynucleotides encoding the polypeptides of the present invention.

Another embodiment of the present invention provides polynucleotides which hybridizes to the foregoing polynucleotides or any complementary strand thereof under stringent condition and encodes the polypeptide of the present invention.

Another embodiment of the present invention provides vector comprising the polynucleotide of the present invention. Another embodiment of the present invention provides host cell carrying the vector of the present invention.

Another embodiment of the present invention provides a method for producing the polypeptide wherein the method comprises the steps of transforming a host cell, in particular an animal cell, with the vector of the present invention, culturing the host cell, and harvesting the polypeptide produced by the host cell. Another embodiment of the present invention provides an antibody, in particular a monoclonal antibody, which is specific to the polypeptide of the present invention.

Another embodiment of the present invention provides an agonist which stimulates collectin activity to be offered by the polypeptide of the present invention, as well as an antagonist which inhibits collectin activity to be offered by the polypeptide of the present invention.

### Effects of the Invention

The present invention provides the ideal novel polypeptides which offer collectin activity and are useful for the elucidation of mechanisms of human immune function and those of a wide variety of diseases such as bacterial infections, and also useful for the development of reagents and drugs for the diagnosis, prophylaxis and therapy on such diseases.

### BRIEF DESCRIPTION OF DRAWINGS

FIG. 1 Schematic view illustrating relationship among collectin and the relative proteins thereof.

FIG. 2 Schematic view illustrating the alignment of the amino acid sequence among the known collectins.

FIG. 3 Schematic view illustrating the structure of hCL-K1 polypeptide.

FIG. 4 Schematic view illustrating the alignment of the amino acid sequences among the known collectin and hCL-K1 polypeptide.

FIG. 5 Experimental result indicating expression of hCL-K1 polypeptide in human tissues.

FIG. 6 Schematic view illustrating the phylogenetic tree of collectins.

FIG. 7 Experimental result indicating expression of hCL-K1 polynucleotide in human tissues.

FIG. 8 Experimental result indicating sugar binding activity in hCL-K1 polypeptide.

FIG. 9 Experimental result indicating hCL-K1 polypeptide purified from serum.

### DESCRIPTION OF SYMBOLS

A - - - Collectin
B - - - Carbohydrate Chain Recognition Domain
C - - - Collagen-Like Domain

### BEST MODE FOR CARRYING OUT THE INVENTION

Merit of the present invention is noted in detail as follows.

The present inventors performed research continuously on any gene encoding expression of human collectin and have successfully cloned the novel collectin genes (hereinafter referred to as ‘hCL-K1 polynucleotide’). Namely, a polypeptide (hereinafter referred to as ‘hCL-K1 polypeptide’) which is an expression product of hCL-K1 polynucleotide and consists of consecutive 271 amino acids set out in SEQ ID NO: 2 has C-terminal side comprising both carbohydrate chain recognition domain (113^{th}-271^{st} amino acids in SEQ ID NO: 2) which is believed to participate in fundamental immunity and collagen-like domain (41^{st}-112^{th} amino acids in SEQ ID NO: 2) consisting of amino acids of (Gly-Xaa-Yaa)ₙ.

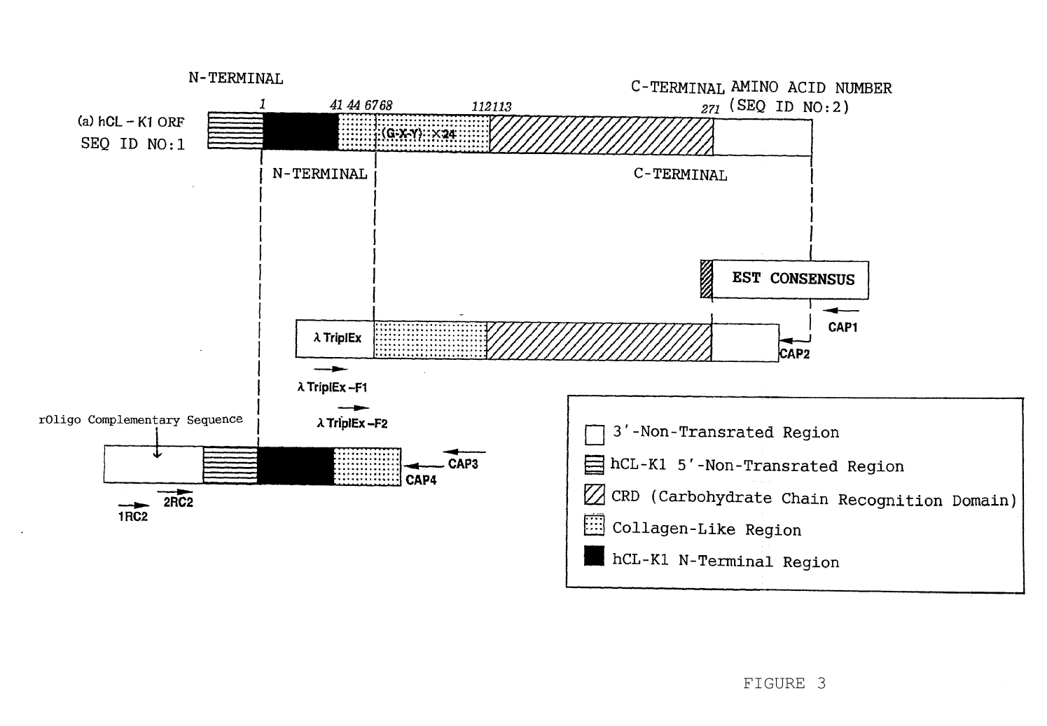

Then, with reference to FIG. 3, the particular amino acids (1^{st}-43^{rd} amino acids) in N-terminal side of hCL-K1 polypeptide had the signal sequence and an amino acid sequence on collagen structure (single coil).

Three mutated polypeptides were generated from hCL-K1 polypeptide though alternative splicing of mRNA and designated them respectively as hCL-K1v1, hCL-K1v2 and hCL-K1v3.

hCL-K1v1 polypeptide consisting of consecutive 223 amino acids set out in SEQ ID NO: 5 and does not have 44^{th}-91^{st} amino acids in SEQ ID NO: 2 (394^{th}-537^{th} nucleotides in SEQ ID NO: 1).

hCL-K1v2 polypeptide consisting of consecutive 247 amino acids set out in SEQ ID NO: 8 and does not have 44^{th}-67^{th} amino acids in SEQ ID NO: 2 (394^{th}-465^{th} nucleotides in SEQ ID NO: 1).

hCL-K1v3 polypeptide consisting of consecutive 247 amino acids set out in SEQ ID NO: 11 and does not have 68^{th}-91^{st} amino acids in SEQ ID NO: 2 (466^{th}-537^{th} nucleotides in SEQ ID NO: 1).

All of such three mutated polypeptides were generated from differential splicing on collagen-like domain in hCL-K1 polypeptide.

Consecutive 271 amino acids set out in SEQ ID NO: 2 is consisting of hCL-K1 polypeptide. Nucleotide sequence consisting of 813 nucleotides encoding such 271 amino acids is set out in SEQ ID NO: 3. Amino acid sequence set out in SEQ ID NO: 2 has typical amino acid sequences on collectin like signal sequence, collagen-like domain and carbohydrate chain recognition domain. Full-length nucleotide sequence encoding such hCL-K1 polypeptide is set out in SEQ ID NO: 1.

Consecutive 223 amino acids set out in SEQ ID NO: 5 is consisting of hCL-K1v1 polypeptide. Nucleotide sequence consisting of 669 nucleotides encoding such 223 amino acids is set out in SEQ ID NO: 6. Amino acid sequence set out in SEQ ID NO: 5 has typical amino acid sequences on collectin like signal sequence, collagen-like domain and carbohydrate chain recognition domain. Full-length nucleotide sequence encoding such hCL-K1v1 polypeptide is set out in SEQ ID NO: 4.

Consecutive 247 amino acids set out in SEQ ID NO: 8 is consisting of hCL-K1v2 polypeptide. Nucleotide sequence consisting of 741 nucleotides encoding such 247 amino acids is set out in SEQ ID NO: 9. Amino acid sequence set out in SEQ ID NO: 8 has typical amino acid sequences on collectin like signal sequence, collagen-like domain and carbohydrate chain recognition domain. Full-length nucleotide sequence encoding such hCL-K1v2 polypeptide is set out in SEQ ID NO: 7.

Consecutive 247 amino acids set out in SEQ ID NO: 11 is consisting of hCL-Kv3 polypeptide. Nucleotide sequence consisting of 741 nucleotides encoding such 247 amino acids is set out in SEQ ID NO: 12. Amino acid sequence set out in SEQ ID NO: 11 has typical amino acid sequences on collectin like signal sequence, collagen-like domain and carbohydrate chain recognition domain. Full-length nucleotide sequence encoding such hCL-K1v3 polypeptide is set out in SEQ ID NO: 10.

Further, the present invention involves with an altered amino acid sequences which are similar to the amino acid sequence of forming hCL-K1 polypeptide and any nucleotide sequence encoding such altered amino acid sequences. Regardless of natural product or artificial product, the altered amino acid sequence is directed to any amino acid sequence wherein one to several amino acids in the consecutive amino acids set out in SEQ ID NOs: 2, 5, 8 or 11 is deleted, substituted or added, nevertheless, it offers collectin activity, for example, an immunological activity that systematically involves with fundamental human immunity to activate complement systems, which is substantially similar to those to be realized by the polypeptide consisting of the consecutive amino acids set out in SEQ ID NOs: 2, 5, 8 or 11. In the meantime, such deletion, substitution or addition of one or several amino acids may be deletion, substitution or addition of amino acids without dramatically changing hydrophilic nature, hydrophobic nature, acidity, basicity and functional group of hCL-K1 polypeptide of the present invention and without substantially changing collectin activity to be offered by calcium ion (Ca²⁺) dependent carbohydrate chain recognition domain or collagen-like domain.

Based on the known amino acid sequence and protein structure on any protein in collectin family, for example, it is believed that deletion, substitution or addition of from about 1 to about amino acids in calcium ion dependent carbohydrate chain recognition domain may be allowed, while from about 1 to about 50, preferably from about 1 to about 15 amino acids in collagen-like domain may be allowed.

Further, the present invention comprises a polynucleotide which hybridizes to the polynucleotide consisting of the nucleotide sequences set out in SEQ ID NO: 1, 4, 7 or 10 or any complementary strand thereof under stringent condition. The ‘stringent’ condition used herein may involve a condition, for example, of incubating in a solution containing 5×SSC, 5% Denhardt's solution (0.1% BSA, 0.1% Ficol 1400, 0.1% PVP), 0.5% SDS and 20 μg/ml denatured sermon sperm DNA at 37° C. overnight followed by a wash with 2×SSC containing 0.1% SDS at room temperature. SSPE may be employed in place of SSC. Thus resultant polynucleotide is speculated to exhibit at least 50% or greater homology to the nucleotide sequences set out in SEQ ID NO: 1, 4, 7 or 10. It is believed that many of the proteins encoded by the polynucleotide which hybridizes under the stringent condition to polynucleotide consisting of the nucleotide sequences set out in SEQ ID NO: 1, 4, 7 or 10 or any complementary strand thereof will offer collectin activity which is similar to those to be offered by hCL-K1 polypeptide. Accordingly, these polypeptides offering such collectin activity are also fallen within the scope of the present invention.

Regardless of natural product or artificial product, the present invention further comprises derivatives of hCL-K1 polypeptide in the form like homologues, mutants, modified forms or polymorphic variants and fragments of these derivatives.

Term ‘homologue’ used herein usually refers to nucleotide sequences or amino acid sequences that bear high homology, which are homologous at least 50% or more, preferably 70% or more, more preferably 90% or more. When some nucleotides or some amino acids are deleted or inserted in such sequence, it is desirable to perform homologous search which allows gap junction. For example, homologues can be searched with a procedure of multiple alignment (trade name: SODHO, Fujitsu Limited). As the algorithm to search homology, Smith-Waterman algorithm is the most accurate tool and may also be employed. Alternatively, FASTA or BLAST may also be utilized via internet.

Term ‘mutant’ used herein usually includes, for example, allele and Single Nucleotide Polymorphism (SNP). Further, any nucleotide sequence mutated due to change of degree on degeneracy of codon is also fallen within the scope of the present invention.

Partial alteration on any codon in the nucleotide sequence may be performed according to the known procedure like the site directed mutagenesis method (Mark, D. F. et al., Proc. Natl. Acad. Sci. USA., 81, 5662, 1984) by employing some primers consisting of synthesized oligonucleotides that encode desired alteration. Artificial gene mutants so made are also fallen within the scope of the present invention. When mutation is beyond the degree on degeneracy of the codon, it is preferable that the mutated amino acid translated by the mutated codon offers function which is similar to that to be offered by normal amino acid. It is preferable that mutation should be performed among similar amino acids in view of their properties, functions or characteristics, for example: the mutation among aliphatic amino acids such as alanine, valine, leucine and isoleucine; the mutation among neutral amino acids such as glycine, alanine, serine, threonine, valine, leucine, isoleucine, cysteine, methionine, phenylalanine, tyrosine, proline, tryptophan, asparagines and glutamine; the mutation among acidic amino acids such as aspartic acid and glutamic acid; the mutation among basic amino acids such as arginine, lysine and histidine; the mutation among amino acids such as serine and threonine having a hydroxyl group respectively; the mutation among amino acids such as phenylalanine and tyrosine having an aromatic ring respectively. These artificially or naturally mutated proteins are also fallen within the scope of the present invention. Site-directed mutagenesis may be realized with PCR method, otherwise, any mutation at any optional site would also be realized by the other known methods.

The term ‘modified form’ used herein may usually be realized through conventional techniques, for example, of acetylation, acylation, ADP-ribosylation, amidation, myristoylation, glycosylation, hydroxylation, phosphorylation, sulfation, formylation, methylation, polyethyleneglycolation, lipid coupling, nucleotide coupling, metal coupling (e.g., calcium addition), fusion with other protein (e.g., albumin) and dimerization. For example, since glycosylation does not occur when the host is in Escherichia coli, the expression may be performed in eucaryotic cells when glycosylation is intended. Besides mammalian cells, insect cells may be also used because glycosylation proceeds post-translationally therein.

The term ‘polymorphic variant’ used herein may usually involves, for example, polymorphisms caused by structural or conformational differences in chromosomal DNA, polymorphisms due to change of a gene into its allelic gene. In general, genes of eucaryotic cells often exhibit polymorphic event, thereby, one or more amino acids are substituted, whilst the protein activity is still retained in spite of such substitution. Therefore, any gene taken by artificially modifying the gene encoding amino acid sequence set out in SEQ ID NO: 2, 5, 8 or 11 through substitution, deletion, addition and/or insertion is fallen within the scope of the present invention as far as the altered protein encoded by such protein express collectin activity. Any altered polypeptide wherein amino acid sequence set out in SEQ ID NO: 2, 5, 8 or 11 is artificially modified would also be fallen within the scope of the present invention as far as it expresses collectin activity.

The term ‘fragment’ used herein may usually refer to any optional fragments derived from the amino acid sequence consisting of hCL-K1 polypeptide, which may include, for example, an extracellular domain, an intracellular domain, a transmembrane domain, a collagen-like domain, a carbohydrate chain recognition domain, a collectin-like domain, a hydrophobic domain (e.g., a transmembrane domain), a hydrophilic domain (domains other than hydrophobic domains), and any fragment obtained through fusion of these fragments. Examples of such fragments are as follows.

With reference to hCL-K1 polypeptide, the amino acid sequence set out in SEQ ID NO:2 has the fragment comprising 113^{th}-271^{st} amino acids which forms a carbohydrate chain recognition domain, the fragment comprising 41^{st}-271^{st} amino acids which forms a carbohydrate chain recognition domain and a collagen-like domain, and the fragment comprising 41^{st}-112^{th} amino acids which forms a collagen-like domain.

With reference to hCL-K1v1 polypeptide, the amino acid sequence set out in SEQ ID NO:5 has the fragment comprising 41^{st}-223^{rd} amino acids which forms a carbohydrate chain recognition domain and a collagen-like domain, and the fragment comprising 41″-64^{th} amino acids which forms a collagen-like domain.

With reference to hCL-K1v2 polypeptide, the amino acid sequence set out in SEQ ID NO:8 has the fragment comprising 41^{st}-247^{th} amino acids which forms a carbohydrate chain recognition domain and a collagen-like domain, and the fragment comprising 41^{st-}88^{th} amino acids which forms a collagen-like domain.

With reference to hCL-K1v3 polypeptide, the amino acid sequence set out in SEQ ID NO:11 has the fragment comprising 41^{st}-247^{th} amino acids which forms a carbohydrate chain recognition domain and a collagen-like domain, and the fragment comprising 41^{st}-88^{th} amino acids which forms a collagen-like domain.

Process for Taking hCL-K1 Polynucleotide

Any method can be employed to produce hCL-K1 polynucleotide of the present invention. For example, the nucleotide sequence encoding hCL-K1 polynucleotide of the present invention can be obtained by preparing mRNA from the cells that are expressing hCL-K1 polypeptide, and converting it into a double stranded DNA by a conventional technique. In order to prepare mRNA, guanidine isothiocyanate calcium chloride method (Chirwin, et al., Biochemistry, 18, 5294, 1979) can be employed. Poly(A)-RNA can be prepared from total RNA by making use of supports with oligo(dT), for example, affinity chromatography with sepharose or latex particles. Such RNA so obtained is employed as a template to treat it with reverse transcriptase under the presence of oligo(dT) or a random primer each of which is complementary to poly(A) chain present at 3′-terminus, or a synthesized oligonucleotide primer corresponding to a part of the amino acid sequence consisting of hCL-K1 polypeptide (Mol. Cell. Biol., 2, 161, 1982; Mol. Cell. Biol., 3, 280, 1983; Gene, 25, 263, 1983). Double stranded cDNA can be obtained by treating cDNA strand so prepared with, for example, E. coli RNaseH, E. coli DNA polymerase 1, E. coli DNA ligase to alter it into the DNA strand. A cDNA library can be produced by incorporating this cDNA into a plasmid vector, a phage vector or a cosmid vector to transform E. coli, otherwise by transfecting it into E. coli following in vitro packaging.

Any plasmid vector can be used as far as it can be replicated and maintained in the host, while any phage vector can also be used as far as it can be proliferated in the host. Available cloning vectors may include pBR322, pUC19, λgt10 and λgt11. Further, when vector is subjected to an immunological screening, it is preferably to use a vector which have a promoter that can express hCL-K1 polynucleotide in the host.

To incorporate cDNA into a plasmid, the process like that of Maniatis et al. (Molecular Cloning, A Laboratory Manual, second edition) can serve as a reference. Further, to incorporate cDNA into a phage vector, the process like that disclosed in Hyunh et al. (DNA cloning, a practical approach, 1, 49, 1985) can serve as a reference.

As the process for introducing the expression vector described above into host cells, some methods, for example, transfection by lipopolyamine method, DEAE-dextran method, Hanahan method, lipofectin method, calcium phosphate method; microinjection, and electroporation (Molecular Cloning, A Laboratory Manual, second edition) may be used. Then, in vitro packaging can be readily performed with commercially available kits (manufactured by Stratagene or Amersham).

In order to isolate cDNA encoding hCL-K1 polypeptide from a cDNA library prepared as described above, any known method for screening cDNA can be used in combination with the other such screening method. For example, a probe labeled with ³²P is produced, and a clone containing the desired cDNA can be screened by a colony hybridization method (Proc. Natl. Acad. Sci. USA, 72, 3961, 1975) or a plaque hybridization method (Molecular Cloning, A Laboratory Manual, second edition, Cold Spring Harbor Laboratory, 2, 108, 1989). Then, any clone may be selected by PCR method. Further, the desired clone can be selected through the use of an antibody that recognizes hCL-K1 polypeptide when cDNA library is produced with a vector that can express the cDNA.

Furthermore, when hCL-K1 polynucleotide is isolated from cells that express hCL-K1 polynucleotide, for example, cells that express gene are dissolved with SDS or proteinase K, followed by a phenol treatment. Unnecessary RNA is digested with ribonuclease. DNA so obtained is digested with restriction enzyme, and DNA fragments so digested are amplified with phage or cosmid to produce a library. Then hCL-K1 polynucleotide can be obtained by selecting the desired clone.

Nucleotide sequence of DNA so obtained can be sequenced by Maxam-Gilbert method (Proc. Natl. Acad. Sci. USA, 74, 560, 1977) or Sanger's method (Proc. Natl. Acad. Sci. USA, 74, 5463, 1977). hCL-K1 polynucleotide can be obtained by excising it from the clone so taken with restriction enzyme.

By making use of the primer synthesized based on the nucleotide sequence of hCL-K1 polynucleotide, cloning can also be performed by RT-PCR method using poly(A)⁺RNA of the cells expressing hCL-K1 polynucleotide as a template. Further, the desired cDNA can also be obtained without depending on PCR by directly screening the cDNA library after producing or synthesizing a probe based on the nucleotide sequence of hCL-K1 polynucleotide. hCL-K1 polynucleotide of the present invention can be selected among numerous genes obtained by the foregoing methods by confirming the nucleotide sequence of such gene. Gene of the present invention can also be produced according to the conventional method known in the art by employing nucleotide chemical synthesis like phosphoimidite method (Mattencci, M. D. et al., J. Am. Chem. Soc., 130, 3185, 1981).

Process for Producing Expression Vector

The present invention also provides a vector comprising a nucleotide sequence of hCL-K1 polynucleotide. The vector is any vector which can express hCL-K1 polypeptide and may includes, for example, a plasmid vector, an RNA vector, a DNA vector, a virus vector and a phage vector. Illustratively available vector thereon may includes pBAD/H is, pRSETA, pcDNA2.1, pTrcHis2A, pYES2, pBlueBac4.5, pcDNA3.1 or pSecTag2 manufactured by Invirtogen, pET or pBAC manufactured by Novagen Co., pGEM manufactured by Promega, pBluescriptll, pBs, Phagescript, pSG or pSV2CAT manufactured by Stratagene, or pGEX, pUC18/19, pBPV, pSVK3 or pSVL manufactured by Pharmacia Co. hCL-K1 polynucleotide ligated to the expression vector is operatively linked to a promoter. The promoter may includes, for example, phage XPL promoter, E. coli lac, trp, tac promoter, SV40 early promoter, SV40 late promoter, T7 promoter, T3 promoter and retrovirus LTR promoter. In particular, the prompter to be used for eukaryotic cells may includes CMV promoter, HSV promoter, SV40 early promoter, SV40 late promoter, retrovirus LTR promoter, RSV promoter and metallothionein promoter. Then the expression vector may contain a marker and an enhancer to allow selection of the transformed host. Illustratively available marker may include dihydrofolate reductase gene, neomycin resistant gene and ampicillin resistant gene. Illustratively available enhancer may include SV40 enhancer, cytomegalovirus early enhancer promoter and adenovirus enhancer.

Process for Producing Transformed Cells

By employing the foregoing vector, the present invention further provides transformed cells carrying a polynucleotide of the present invention incorporated into the vector which allows the expression of the polynucleotide. Host cell for the transformed cell of the present invention may include any cell (including microorganisms) which can express hCL-K1 polynucleotide incorporated into the expression vector of the present invention. But, of those, animal cells and insect cells are preferable.

Illustratively preferable animal cells or insect cells may include cells from mammalian like human, hamster or rat, or cells from insect like fly or silkworm. For example, CHO cells, COS cells, BHK cells, Vero cells, myeloma cells, HEK293 cells, HeLa cells, Jurkat cells, mouse L cells, mouse C127 cells, mouse FM3A cells, mouse fibroblast, osteoblast, chondrocyte, S2, Sf9, Sf21, High Five™ cells may be available. Then, Escherichia coli or Saccharomyces cerevisiae is preferable as microorganism. Vector can be introduced into such hosts according to the foregoing method.

Cells expressing hCL-K1 polypeptide can be used to analyze collectin pathway involving in infectious diseases or immunity. Then, those cells can be utilized in producing hCL-K1 polypeptide itself or hCL-K1 polypeptide having carbohydrate chain. Further, this expression cell is able to use in screening for obtaining an agonist or an antagonist to hCL-K1 polypeptide.

Process for obtaining hCL-K1 polypeptide

The present invention also provides a process for producing hCL-K1 polypeptide which comprises the steps of culturing the foregoing transformed cells and harvesting hCL-K1 polypeptide produced by such transformed cells. Cultivation of cells, isolation and purification of the polypeptide may be performed according to any methodology known in the art.

The hCL-K1 polypeptide of the present invention can be expressed as a recombinant fusion protein to easily isolate, purify and recognized the polypeptide. The recombinant fusion protein is a protein expressed by adding an appropriate peptide chain to the N-terminal end and/or C-terminal end of a protein expressed by nucleotide sequence encoding the target protein. In order to purify smoothly the expressed protein, it may be expressed as a fusion protein having a signal on extracellular secretion. Further, the protein can be obtained from several kinds of protein sources like cultured cells, cultured tissues or transformed cells with any methodology known in the art, for example, purification methods like salting out such as ammonium sulfate precipitation technique, gel filtration technique using Sephadex, ion exchange chromatographic technique, hydrophobic chromatographic technique, dye gel chromatographic technique, electrophoresis technique, dialysis, ultrafiltration technique, affinity chromatographic technique and high performance liquid chromatographic technique.

Method for Utilizing hCL-K1 Polynucleotide

Probes for detecting hCL-K1 polynucleotide can be designed based on the nucleotide sequence set out in SEQ ID NO: 3, 6, 9 or 12. Primers can also be designed to amplify DNA or RNA including these nucleotide sequences.

To design a probe or a primer based on the particular sequence is often performed by one skilled in the art. Oligonucleotide having the designed nucleotide sequence can be obtained through chemical synthesis. Oligonucleotide having an appropriate label can be used in several forms of hybridization assay. Alternatively, it can be used in a reaction, like PCR, for synthesizing nucleotides. Oligonucleotide to be used as a primer is of at least about 10 nucleotides in length, and preferably of about 15 to about 50 nucleotides in length, while that to be used as a probe is of from about 100 nucleotides to full length thereof. Then, they can also be used for diagnosis of diseases caused by mutation in hCL-K1 polynucleotide, because such primer or probe can be used for detecting genetic mutation encoding hCL-K1 polypeptide and for detecting SNP. They are expected to be available for diagnosis of diseases like bacterial infections. Further, they are also useful for gene therapy whereby hCL-K1 polynucleotide is introduced into a living body to allow the expression thereof.

Further, it is also possible to take from a genome a promoter region and an enhancer region of hCL-K1 polynucleotide based on nucleotide sequence of hCL-K1 polynucleotide of the present invention. In particular, these control regions can be taken by a method according to those disclosed in Japanese Patent Provisional Publication No. 6-181767, J. Immunol., 155, p. 2477 (1995), and Proc. Natl. Acad. Sci, USA., 92, p. 3561 (1995). The term ‘promoter region’ referred to herein is usually directed to a DNA region which controls expression of a gene in upstream of a transcription initiation site, while that ‘enhancer region’ referred to herein is directed to a DNA region that enhances expression of a gene in an intron, 5′-untranslated region or a 3′-untranslated region.

Method for Utilizing hCL-K1 Polypeptide

hCL-K1 polypeptides of the present invention can be utilized to elucidate mechanisms of fundamental human immunity and of development on various diseases such as bacterial infections. They can then be used to develop reagents and drugs to be employed for the diagnostic, prophylactic and therapeutic methods on such diseases.

Further, they can be used as an antigen for producing antibodies to hCL-K1 polypeptide.

Additionally, they can be utilized in a process for the screening of an agonist or an antagonist.

Agonist and Antagonist

The present invention provides agonists which stimulate the collectin activity of hCL-K1 polypeptide and an antagonists which inhibit the collectin activity of hCL-K1 polypeptide.

As a method for screening the antagonist, for example, a competitive experimental system, namely, a system of contacting cells expressing hCL-K1 polypeptide to a candidate inhibitor and mannose or an antibody can be used. Candidate inhibitor is then screening based on the binding ratio to the mannose. Conventionally methods well known in the art would also be performed for screening such antagonist. Then, the antagonists of the present invention may further include antisense nucleotide that inhibits the expression of hCL-K1 polynucleotide. As the other screening methods, there is a method for measuring a change in extracellular pH due to activation of a receptor (Science, 246, pp. 181-296 (1989)).

Transgenic Non-Human Animal

The present invention provides transgenic non-human animals having an altered expression level of hCL-K1 polynucleotide. Form of hCL-K1 polynucleotide may include any DNA, i.e., cDNA, genomic DNA or synthesized DNA encoding hCL-K1 polypeptide. In order to express hCL-K1 polynucleotide, both steps of transcription and translation are performed. The transgenic non-human animals of the present invention are useful for investigation of functions or expression mechanisms of hCL-K1 polypeptide, elucidation of mechanisms of diseases that may be involved with hCL-K1 polynucleotide, development of diseased animal models to be used for screening medicine and for performing safety tests of the same.

hCL-K1 polynucleotide can be artificially modified to increase or decrease the expression level in comparison with the native expression level thereof by introducing mutation such as deletion, substitution, addition and/or insertion into a part of some important sites (e.g., enhancer, promoter, intron) that appropriately regulate the expression of hCL-K1 polynucleotide.

The introduction of such mutation can be performed by known methods, thereby, a transgenic animal can be realized. Transgenic animals in their narrow means refer to those having germ cells into which a foreign gene was artificially introduced by a genetic recombination technique. In their broader means, they refer to those having a chromosome with a foreign gene introduced stably therein at an early stage of the development of the individual and having a genotype that can be transmitted to the progeny thereof, and may include antisense transgenic animals having a particular gene of which function was suppressed using an antisense RNA, knockout animals having a particular gene knocked out using embryonic stem cells (ES cell), and animals having point mutation of DNA introduced, namely, all of them are animals.

Term ‘transgenic animals’ used herein may usually include all vertebrates other than human. The transgenic animals of the present invention are useful for investigation on functions or expression mechanisms of hCL-K1 polypeptide, elucidation on mechanisms of diseases that are involved in human cells expressing hCL-K1 polypeptide, development of diseased animal models to be used in screening of medicine and in performing safety tests on the same.

Method for producing a transgenic mouse may include a process in which a gene is directly introduced into a nucleus of an ovum in a anterior nucleus phase with a micropipette under a phase contrast microscope (microinjection technique, U.S. Pat. No. 4,873,191) and a process in which embryonic stem cells (ES cells) are used. On the other hand, the other processes are also developed and may include a process in which a gene is introduced into a retrovirus vector or an adenovirus vector followed by infection into an ovum and a sperm vector technique in which a gene is introduced into an ovum via a sperm.

The sperm vector technique is a genetic recombinant process in which a foreign gene is attached to a sperm, or a foreign gene is introduced into a sperm cell with an electroporation technique, and then the foreign gene is introduced into an ovum by fertilizing the ovum (M. Lavitranoet et al., Cell, 57, 717, 1989). Alternatively, in vivo site directed genetic recombination like a cre/loxP recombinase system of bacteriophage P1 and a FLP recombinase system of Saccharomyces cerevisiae may also be employed. Then a method has been also reported in which a transgene of a desired protein is introduced into a non-human animal with retrovirus.

Method for producing a transgenic animal with a microinjection technique is performed, for example, as described below.

First of all, it is necessary to prepare a transgene which is substantially consisted of a promoter involved in expression control, a gene encoding the particular protein and a poly(A) signal. The manner of the expression and/or the expression level of the particular molecule may be changed with the promoter activity. Then, because transgenic animals are different among the produced lineages in respect to copy number on the introduced transgene or the introduced site in the chromosome, the manner of the expression and/or the expression level must be confirmed for each of the lineages. Since it has been elucidated that the expression level is altered depending on the untranslated region or splicing, an intron sequence to be spliced at a preceding site of poly (A) signal may be previously introduced. It is important to use a gene, which is introduced into a fertilized ovum, has as high purity as possible. The animal to be used may include mice for use in collecting fertilized ova (5-6 weeks old), male mice for use in mating, female pseudopregnant mice and vas deferens ligated male mice.

In order to efficiently take the fertilized ova, gonadotropin or the like may be used for inducing the ovulation. The fertilized ova are harvested, and a gene in an injection pipette is introduced into a male pronucleus of the ovum by a microinjection technique. An animal (e.g., a pseudopregnant mouse) for use in repositioning the injected ova to an oviduct is provided, and about 10-about 15 ova are transplanted per one animal. Thereafter, the newborn mouse can be examined as to whether or not that the transgene is actually introduced by extracting genomic DNA from the end portion of the tail, and detecting the transgene by a Southern method or a PCR technique, alternatively with a positive cloning technique where a marker gene to be activated upon only the occurrence of homologous recombination is inserted. Further, in order to confirm expression of the transgene, a transcription product derived from the transgene is detected by a Northern method or a RT-PCR technique. Alternatively, a western blotting method may be performed with a specific antibody to the protein or a fragment thereof.

Knockout Mouse

The knockout mouse of the present invention is that treated to deprive the function of hCL-K1 polynucleotide. Knockout mouse is a transgenic mouse in which an arbitrary gene is destroyed by a homologous recombination technique to impair the corresponding function. The knockout mouse can be produced by homologous recombination using ES cells, followed by the selection of the embryonic stem cell having one of the allelic gene altered/destroyed. For example, a chimeric mouse (chimera is a single individual built-up with somatic cells based on more than two fertilized ova) having cells derived from the embryonic stem cells and cells derived from the embryo being mixed may be taken by injecting the embryonic stem cell that had been genetically engineered at blastocyst stage or morulae stage of the fertilized ovum. When this chimeric mouse is crossbred with a normal mouse, it would be possible to produce a heterozygotic mouse wherein one of the allelic gene is entirely altered/destroyed. Further, a homozygotic mouse can be produced by crossbreeding heterozygotic mice each other.

Homologous recombination is recombination relied on a mechanism of genetic recombination between two genes having identical or extremely similar base sequences. For the selection of cells with the homologous recombination, PCR can be employed. PCR reaction employing primers corresponding to a part of the inserted gene and a part of the region expected to be inserted may be performed to reveal the homologous recombination occurring in cells that could yield the amplification products. Also, when the homologous recombination is due to a gene expressed in embryonic stem cells, the gene to be introduced may be joined to a neomycin resistant gene to allow the selection after the introduction into cells by making them resistant to neomycin. Accordingly, conventional methods known in the art and the modified methods thereof can be employed to enable easy selection.

Method of Producing Antibodies

The present invention further provides antibodies that recognize hCL-K1 polypeptide or fragments thereof.

Antibodies of the present invention include, for example, those to hCL-K1 polypeptide consisting of consecutive amino acids set out in SEQ ID NO: 2, 5, 8 or 11 or a fragment thereof. The antibodies (e.g., polyclonal antibodies, monoclonal antibodies, peptide antibodies) or antisera to hCL-K1 polypeptide or a fragment thereof can be produced using hCL-K1 polypeptide or a fragment thereof of the present invention as an antigen according to any method known in the art for producing the antibodies or antisera. In particular, antibodies that can control the function of hCL-K1 polypeptide (e.g., antibodies that recognize carbohydrate chain recognition domain and a collagen like domain) are useful for medicine containing such antibodies.

hCL-K1 polypeptide or a fragment thereof of the present invention may be administered neat or with a diluent or a carrier to a warm-blooded animal at a site that enables the production of the antibody upon the administration. In order to facilitate the production of antibodies upon the administration, complete Freund's adjuvant or incomplete Freund's adjuvant may be administered. The administration may be usually performed once per 1 to 6 weeks, and two to ten times in total. The warm-blooded animal to be used may include, for example, monkey, rabbit, dog, guinea pig, mouse, rat, sheep, goat and chicken. Of these, mouse and rat may be preferably used. Wistar and SD strain rat are preferably used as a rat, while BALB/c, C57BL/6 and ICR strain mouse are preferably used as a mouse.

Upon the production of cells that produce a monoclonal antibody, an individual with the antibody titer that can be recognized therein is selected from the warm-blooded animals e.g., mice that had been immunized with an antigen. On two to five days after final immunization, spleen or lymph node is collected, and the antibody producing cells contained therein are subjected to the fusion with myeloma cells to prepare monoclonal antibodies producing cells. Antibody titer in the antiserum may be determined, for example, by subjecting a labeled hCL-K1 polynucleotide described below to a reaction with the antiserum, and thereafter measuring the activity of the label bound to the antibody. Such fusion operation can be performed in accordance with a known technique, for example, the process of Ko\ddot{h}ler and Milstein (Nature, 256, p. 495 (1975)) and the modified process thereof (J. Immunol. Method, 39, p. 285 (1980); Eur. J. Biochem., 118, p. 437 (1981); Nature, 285, p. 446 (1980)). Examples of the fusion accelerating agent may include polyethylene glycol (PEG) and Sendai virus. Of these, polyethylene glycol may be preferably used. In order to raise the efficiency of the fusion, lectin, poly-L-lysine or DMSO may be added optionally.

Examples of the myeloma cell include, for example, X-63Ag8, NS-1, P3U1, SP2/0 and AP-1. Of these, SP2/0 may be preferably used. Ratio of antibody producing cell (spleen cell) number to myeloma cell number preferably used is about 1:about 20-about 20:about 1. PEG (preferably, PEG1000-PEG6000) is added at about 10%-about 80%, then the fusion mixture is incubated at about 20° C.-about 40° C., preferably at about 30° C.-about 37° C. for about 1 minute-about 10 minutes to smoothly fuse cells. There are some screening methods for hybridoma that produces antibodies to hCL-K1 polypeptide. For example, it may include a process wherein a supernatant of hybridoma culture is added to a solid phase (e.g., a microplate) adsorbed with hCL-K1 polypeptide (antigen) directly or with a carrier, and then protein A or an anti-immunoglobulin antibody (when the cells used for the cell fusion was derived from a mouse, anti-mouse immunoglobulin antibody may be used) that was labeled with a radioactive substance or enzyme is added thereto, thereby, detecting the antibody (to hCL-K1 polypeptide) bound to the solid phase. Otherwise, a process in which a supernatant of hybridoma culture is added to a solid phase adsorbed with an anti-immunoglobulin antibody or protein A, and then hCL-K1 polypeptide labeled with a radioactive substance or enzyme is added thereto, thereby, detecting the monoclonal antibody bound to the solid phase and are specific to hCL-K1 polypeptide.

Selection and cloning of the monoclonal antibody to hCL-K1 polypeptide can be performed by any of known methods or the modified methods thereof. Usually, such selection and cloning are performed in a medium for animal cells added with HAT (hypoxanthine, aminopterin and thymidine). The medium for use in the selection, cloning and growing may be any one of the media in which hybridoma can grow. For example, RPMI medium containing about 1%-about 20%, preferably about 10%-about 20% of fetal bovine serum, GIT medium containing about 1%-about 10% of fetal bovine serum, or serum free medium for hybridoma culture. The temperature of the culture may be preferably about 37° C. The culture period may be usually five days to three weeks, preferably one week to two weeks. The culture is usually performed in the presence of 5% carbon dioxide gas. The antibody titer of the supernatant of the hybridoma culture can be measured in a similar manner to measure the antibody titer in an antiserum as described above. Namely, a radioimmunoassay (RIA) technique, an enzyme linked immunosorbent assay (ELISA) technique, a FIA (fluorescent immunoassay) technique, a plaque measurement technique and an agglutination reaction technique may be employed as the measurement process. But, of these, the ELISA technique is preferred used.

Screening according to ELISA technique can be performed in accordance with the following procedure. A protein prepared by a similar process to that for the immunoantigen is immobilized on the surface of each well of an ELISA plate. Next, BSA, MSA, OVA, KLH, gelatin or skimmed milk is immobilized for the purpose of preventing non-specific adsorption. Supernatant solution of the hybridoma culture is added to each well and the immunoreaction is allowed by standing for a predetermined time. Each well is washed using a washing solution such as PBS. Surfactant may be preferably added to this washing solution. An enzyme-labeled secondary antibody is added, and the mixture is allowed to stand for a predetermined time. The enzyme for labeling may includes β-galactosidase, alkaline phosphatase or peroxidase. After such washing step with the same washing solution, enzyme reaction is effected by adding a substrate solution of the labeled enzyme that was employed.

When the desired antibody is present in the supernatant solution of the hybridoma culture so added, the enzyme reaction proceeds and the color of the substrate solution would be changed.

Cloning can be usually performed by any of known methods like a semisolid agar technique or a limiting dilution technique. In particular, after the well in which the desired antibody is produced is confirmed by the process described above, a single clone is taken through such cloning. Cloning is performed preferably with a limiting dilution technique wherein hybridoma cells are diluted and cultured so that one colony per one well of a culture plate is formed. Cloning according to a limiting dilution technique may be performed through the use of feeder cells to elevate the colony formation ability, otherwise, an addition of a cell growth factor such as interleukin 6. Alternatively, FACS and single cell manipulation techniques can be employed for the cloning. The cloned hybridoma is cultured preferably in a serum free medium, and an appropriate amount of the antibody is added to the supernatant thereof. Thus resulting single hybridoma may be subjected to a large scale culture using a flask or a cell culture equipment, or may be cultured in the peritoneal cavity of an animal (J. Immunol. Meth., 53, 313, 1982) to produce a monoclonal antibody. When the culture is performed in a flask, a medium for cell culture (IMDM, DMEM, RPMI 1640 and MEM) containing 0%-about 20% of FCS can be used. When the culture is performed in the peritoneal cavity of an animal, an animal of the same species or the same strain as that from which myeloma cells were derived for the cell fusion, otherwise, an athymic nude mouse may be preferably used. Hybridoma is transplanted after mineral oil such as pristine is previously administered to the animal. After one to two weeks later, the myeloma cells enough proliferate and ascites containing the monoclonal antibody can be taken.

The monoclonal antibody of the present invention is taken by selecting antibody recognizing an epitope specific to hCL-K1 polypeptide and does not cross-react with other polypeptide. In general, an epitope consisting of serial amino acid residues of at least five or more amino acids, preferably, 7 to 20 amino acids in the amino acid sequence constituting the subjected polypeptide is said that it is an epitope inherent in such polypeptide. Therefore, the monoclonal antibody that recognizes a polypeptide consisting of consecutive amino acids set out in SEQ ID NO: 2, 5, 8 or 11 or an epitope consisting of at least five consecutive amino acid residues in such fragments may be identified as the monoclonal antibody specific to hCL-K1 polypeptide of the present invention. When the particular amino acid sequence that is conserved in the consecutive amino acids set out in SEQ ID NO: 2, 5, 8 or 11 is chosen, an epitope which is in common with hCL-K1 polypeptide can be taken. It is then also possible to select a monoclonal antibody that can discriminate each protein by selecting a region including an amino acid sequence specific to each of such amino acid sequences.

Similar to the usual separation and purification process for polyclonal antibodies, separation and purification of the monoclonal antibody to hCL-K1 polypeptide can be performed according to the methodology on separation and purification for an immunoglobulin. Available known purification process may include, for example, a salt precipitation technique, an alcohol precipitation technique, an isoelectric point precipitation technique, an electrophoretic technique, an ammonium sulfate precipitation technique, an adsorption/desorption technique by an ion exchanger (e.g., DEAE), an ultracentrifugation technique, a gel filtration technique, and a specific purification technique in which an antibody alone is collected by an antigen-bound solid phase or an active adsorbent such as protein A or protein G, followed by dissociation of the unnecessary binding to produce the antibody. In order to prevent formation of aggregates or decrease in the antibody titer in the purification step, for example, human serum albumin may be added at a concentration of about 0.05%-about 2%. Otherwise, amino acids such as glycine or α-alanine, in particular, basic amino acid such as lysine, arginine or histidine, saccharides such as glucose or mannitol, salts such as sodium chloride may also be added. Regarding IgM, since it is known to be liable to agglutinate, it may be treated with β-propionolactone and acetic anhydride.

The polyclonal antibody of the present invention can be produced by known methods or the modified methods thereof. For example, in order to produce a polyclonal antibody, an immunoantigen (a polypeptide antigen) itself or a complex formed with the immunoantigen and a carrier protein is used for the immunization of a warm-blooded animal in a similar manner noted in the process for producing the monoclonal antibody described above, followed by collecting from the warm-blooded animal the preparation containing the antibody to hCL-K1 polypeptide of the present invention or a fragment thereof, and then the antibody is purified/isolated. With regard to the complex of an immunoantigen and a carrier protein for immunization of the warm-blooded animal, kind of the carrier protein and mixing ratio of the carrier and hapten may be optionally determined as long as the antibody can be efficiently produced to the hapten subjected to the immunization after crosslinking with the carrier. Thus any kind of the carrier protein may be crosslinked at any ratio. For example, about 0.1-about 20, preferably about 1-about 5 of bovine serum albumin, bovine thyroglobulin or hemocyanin is coupled with 1 of hapten by weight may be used. Then, various condensing agents may be used for the coupling of hapten and carrier, and they may include glutaraldehyde and carbodiimide, and active ester reagents containing maleimide active ester, thiol group or dithiopyridyl group. The condensation product is administered neat or with a carrier or a diluent to a warm-blooded animal at a site that enables the production of the antibody upon the administration. In order to facilitate the production of antibodies upon the administration, complete Freund's adjuvant or incomplete Freund's adjuvant may be administered. The administration may be usually performed once per 2 to 6 weeks, and three to ten times in total. Polyclonal antibodies can be collected from the blood and ascites, preferably from the blood of the warm-blooded animal immunized by a process as described above.

Antibody titer in antiserum can be measured in a similar manner to that for the antibody titer in the antiserum as described above.

Similar to the separation and purification process for monoclonal antibodies, separation and purification of the polyclonal antibody can be performed according to the methodology on separation and purification for an immunoglobulin.

Method of Utilizing Antibodies

Monoclonal antibodies and polyclonal antibodies to hCL-K1 polypeptide or a fragment thereof can be utilized in diagnosis and therapy of the diseases relating to the cells that are expressing hCL-K1 polypeptide. hCL-K1 polypeptide or a fragment thereof can be measured using these antibodies based on immunological binding with hCL-K1 polypeptide or the fragment thereof of the present invention. In particular, the method for measuring hCL-K1 polypeptide or a fragment thereof with such antibody may include, for example, sandwich techniques wherein a sandwich complex is produced by subjecting hCL-K1 polypeptide or a fragment thereof to a reaction with an antibody coupled to an insoluble support and a labeled antibody and detecting the same. Otherwise, it may also include competitive techniques wherein hCL-K polypeptide or a fragment thereof in a sample is measured by subjecting labeled hCL-K1 polypeptide and hCL-K1 or a fragment thereof in a sample to a competitive reaction with the antibody followed by measurement of hCL-K1 polypeptide or a fragment thereof in a sample from the amount of the labeled antigen that reacted with the antibody.

Upon the measurement of hCL-K1 polypeptide or a fragment thereof by the sandwich technique, available methods may include two-step methods in which hCL-K1 polypeptide or a fragment thereof is firstly subjected to a reaction with an immobilized antibody, thereafter, unreacted materials are completely removed by washes, and a labeled antibody is added thereto to form the immobilized antibody-labeled hCL-K1 polypeptide, and one-step methods in which an immobilized antibody, a labeled antibody and hCL-K1 polypeptide or a fragment thereof are mixed concurrently.

Insoluble support for use in the measurement may include, for example, synthetic resin such as polystyrene, polyethylene, polypropylene, polyvinyl chloride, polyester, polyacrylic acid ester, nylon, polyacetal or fluorine-contained resin, polysaccharides such as cellulose or agarose, glasses and metals. Forms of the insoluble support are varied and may include tray-like, spherical, fibrous, cylindrical, discal, vessel-like, cell-like or tubular. The support onto which the antibody had been adsorbed may be stored appropriately in cold in the presence of an antiseptic agent such as sodium azide.

For the immobilization of the antibody, known chemical coupling processes or physical adsorption processes may be employed. Chemical coupling process may include, for example, processes in which glutaraldehyde is used, maleimide processes in which N-succinimidyl-4-(N-maleimidemethyl)cyclohexane-1-carboxylate and N— succinimidyl-2-maleimide acetate are used, carbodiimide processes in which 1-ethyl-3-(3-dimethylaminopropyl) carbodiimide hydrochloride is used. Other process may include maleimidebenzoyl-N-hydroxysuccinimide ester processes, N-succimidyl-3-(2-pyridylthio) propionic acid processes, bisdiazobenzidine processes and dipalmityl lysine processes. Alternatively, a complex that had been formed previously by subjecting the substance to be detected to a reaction with two kinds of antibodies of which epitopes are different can be captured by the third antibody to the antibody which had been immobilized in a similar manner to those described above.

Labeling material may include enzyme, fluorescent materials, luminescence materials, radioactive materials and metal chelates.

Examples of enzyme may include peroxidase, alkaline phosphatase, β-D-galactosidase, malate dehydrogenase, staphylococcus nuclease, delta-5-steroid isomerase, α-glycerolphosphate dehydrogenase, triose phosphate isomerase, horseradish peroxidase, asparaginase, glucose oxidase, ribonuclease, urease, catalase, glucose-6-phosphate dehydrogenase, glucoamylase and acetylcholine esterase.

Examples of fluorescent materials may include fluorescein isothiocyanate, phycobilin protein, rhodamine, phycoerythrin, phycocyanin, allophycocyanin and orthophthalic aldehyde.

Examples of luminescence materials may include isoluminol, lucigenin, luminol, aromatic acridinium esters, imidazole, acridinium salts and modified esters thereof, luciferin, luciferase and aequorin.

Examples of radioactive materials may include ¹²⁵I, ¹²⁷I, ¹³¹I, ¹⁴C, ³H, ³²P and ³⁵S. In addition, low molecular weight hapten such as biotin, dinitrophenyl, pyridoxal or fluorescamine may be conjugated to the antibody. Preferably, horseradish peroxidase may be used as a labeling enzyme. This enzyme can react with many kinds of substrates and can be readily conjugated to the antibody according to a periodic acid method.

When an enzyme is used as a labeling agent, a substrate for measuring their activity, and a color-developing agent as needed may also be employed. When peroxidase is used as an enzyme, hydrogen peroxide (H₂O₂) may be used as a substrate solution, and 2,2′-azino-di-[3-ethylbenzthiazolin sulfonate]ammonium (ABTS), 5-aminosalicylic acid, orthophenylenediamine, 4-aminoantipyrine or 3,3′,5,5′-tetramethylbenzidine may be used as a color-developing agent. When alkaline phosphatase is employed as an enzyme, orthophenylphosphate or paranitrophenylphosphate may be used as a substrate. Alternatively, when β-D-galactosidase is used as an enzyme, fluorescein-di-β-D-galactopyranoside), or 4-methyl-umbelliferyl-β-D-galactopyranoside may be used as a substrate. The present invention also provides a kit equipping the foregoing monoclonal antibody, the polyclonal antibody and some reagents.

Available crosslinking agents may include N,N′-orthophenylenedimaleimide, 4-(N-maleimidemethyl)cyclohexanoyl N-succinimide ester, 6-maleimidehexanoyl N-succinimide ester, 4,4′-dithiopyridine and other known crosslinking agents. The reaction between such a crosslinking agent with the enzyme and the antibody may be performed in accordance with known methods depending upon the properties of the respective crosslinking agents. Additionally, the antibodies to be used may be any fragments thereof, for example, Fab′, Fab, F(ab′)₂ depending on the condition. Further, enzymatically labeled antibodies may be prepared by using a similar process regardless of polyclonal antibodies or monoclonal antibodies. When the enzymatically labeled antibody that was taken with the foregoing crosslinking agent is purified by any known method such as affinity chromatography, more sensitive immunological determination system can be realized. Purified and enzymatically labeled antibody is stored in a cold and dark place after adding thimerosal or glycerol as a stabilizer, alternatively, after being lyophilized.

The subject sample for the measurement may be any sample containing hCL-K1 polypeptide which may include body fluids such as plasma, serum, blood, urine, tissue fluid and cerebrospinal fluid, various types of cells and tissues.

Method for Producing Humanized Antibody

It is ethically impermissible to produce antibodies by immunizing human with an optional antigen. Further, when a mouse monoclonal antibody is administered to a human body, there is a risk of the occurrence of a variety of adverse effects, because the antibody is a heterogeneous protein to human. Therefore, an antibody with lowered antigenicity to human is preferred when the antibody is administered to human.

Besides cell fusion techniques, there are some method like that for producing human monoclonal antibodies involves transformation techniques with Epstein-Barr virus (EBV), fusion techniques in which thus transformed cells and parent cells are fused, and those in which a chimeric antibody or a humanized antibody is produced through genetic engineering techniques. Chimeric antibody is an antibody that was produced by linking immunoglobulin gene fragments from heterogeneous animals. Then, humanized antibody is an antibody having a substituted primary structure in part other than a complementarity determining region (CDR) of H chain and L chain with the corresponding primary structure of a human antibody through introducing the alteration to a mouse antibody or the like that is heterogeneous to human.

For the production of a chimeric antibody, a mouse is immunized first, then an antibody variable region (V region) that binds to an antigen is excised from a gene of the mouse monoclonal antibody, and the V region is linked to a gene of an antibody constant region (C region) derived from human myeloma to produce a chimeric gene. Upon expression of this chimeric gene in a host cell, human-mouse monoclonal antibody can be produced. Because chimeric antibodies are less antigenic to human, they can be utilized as a monoclonal antibody for therapeutic use to be administered into a human body, or for use in diagnostic imaging. Conventional techniques relevant to chimeric antibodies were disclosed in Japanese Patent Provisional Publication No. 05-304989, Japanese Patent Provisional Publication No. 04-330295, WO 9106649, Japanese Patent Provisional Publication No. 63-036786 and Japanese Patent Provisional Publication No. 06-98021.

Then, humanized antibodies were recently developed, which are appreciated as being more useful than chimeric antibodies. Humanized antibody is an antibody that is humanized as a whole molecule except for complementarity determining region of an antibody molecule by grafting only a sequence of a gene for an antigen-binding site (CDR: complementarity determining region) of an antibody molecule into a gene of a human antibody (CDR grafting). This antibody is appreciated as being safer with less antigenicity than the human-mouse chimeric antibody because it has less part derived from a mouse antibody. When SHM-D 33 strain (ATCC CRL 1668) or RF-S1 strain, both of which being human/mouse heteromyeloma, is used as a parent cell for producing a human monoclonal antibody, higher fusion efficiency can be realized that is equivalent to mouse parent cells. Hybridoma that was obtained using these parent cells can be cloned without feeder cells, and it can produce IgG type antibody in a comparatively stable manner in large amount. For the culture of the parent cells, ERDF medium supplemented with 15% FCS may be used, while other operation may be performed similarly to the operation for the murine cells. Additionally, in order to produce an IgG type human monoclonal antibody, human lymphocytes collected from peripheral blood and were sufficiently sensitized with an antigen may be preferably employed. When it is difficult to obtain sufficiently sensitized lymphocytes, sensitization with an antigen may be also performed in vitro. In Japan, clinical trials have been currently performed for humanized antibodies to adult T cell leukemia. With regard to the production of human antibodies and the related art, for example, reference should be made to those disclosed in Genentech Inc., USA (WO 9222653, WO 9845332, WO 9404679, WO 9837200, WO 9404679) and Celltech Inc., England (WO 9429451, WO 9429351, WO 9413805, WO 9306231, WO 9201059, WO 9116927, WO 9116928, WO 9109967, WO 8901974, WO 8901783).

Using the foregoing methods, the antibodies of the present invention can be humanized.

Composition

hCL-K1 polynucleotides or hCL-K1 polypeptide can be used to develop reagents and drugs to be employed for the diagnostic, prophylactic and therapeutic methods on diseases including bacterial infections.

Pharmaceutical composition of the present invention may comprise hCL-K1 polynucleotides or hCL-K1 polypeptide, substances that stimulate or inhibit the activity or activation of hCL-K1 polypeptide, substances including antibodies to hCL-K1 polypeptide and the like (related substance).

These substances can be used neat or after subjecting to several kinds of treatment such as dilution in water. Then they are blended in pharmaceutical products and quasi drugs. In these cases, the amount of the substance to be blended may be determined appropriately. When the substance is formulated for the systemic administration, about 0.001%-about 50% by weight, preferably, about 0.01%-10% by weight is usually permissible.

When the amount is less than about 0.001% by weight, sufficient action of lacrimation may not be enabled. When the amount is greater than about 50% by weight, properties such as stability or flavor of the composition itself may be deteriorated.

Administration route can be optionally selected from the administration via mucosa, transdermal administration, intramuscular administration, subcutaneous administration, endorectal administration and topical ocular administration, in addition to oral administration and intravenous administration described above.

These related substance may be included in the formulation as a salt.

Pharmaceutically acceptable salts may include salts with base such as inorganic base or organic base, acid addition salts such as those of inorganic acid, organic acid, basic or acidic amino acid.

Inorganic bases may include alkaline metal such as sodium, potassium, alkaline earth metal such as calcium and magnesium, and aluminum and ammonium.

Organic bases may include primary amines such as ethanolamine, secondary amines such as diethylamine, diethanolamine, dicyclohexylamine and N,N′-dibenzylethylenediamine, tertiary amines such as trimethylamine, triethylamine, pyridine, picoline and triethanolamine.

Inorganic acids may include hydrochloric acid, hydrobromic acid, nitric acid, sulfuric acid and phosphoric acid.

Organic acids may include formic acid, acetic acid, lactic acid, trifluoroacetic acid, fumaric acid, oxalic acid, tartaric acid, maleic acid, benzoic acid, citric acid, succinic acid, malic acid, methanesulfonic acid, ethanesulfonic acid, benzenesulfonic acid and p-toluenesulfonic acid.

Basic amino acids may include arginine, lysine and ornithine. Acidic amino acids may include aspartic acid and glutamic acid.

Available dosage forms for use in oral administration may include powdered formulations, granulated formulations, encapsulated formulations, pills, tablets, elixirs, suspensions, emulsions and syrups. Such formulations may further be modified through release control, stabilization, facilitation of disintegration, blocking of disintegration, enteric coating or facilitation of absorption.

Available dosage forms for the intraoral topical administration may include chewable formulations, sublingual formulations, buccal formulations, lozenges, ointments, plasters and liquid formulations. Such formulations may further be modified through release control, stabilization, facilitation of disintegration, blocking of disintegration, enteric coating and facilitation of absorption.

Drug delivery system (DDS) techniques well known in the art may be applied to dosage forms as described above. Term ‘DDS formulation’ used herein may usually directed to formulations that are prepared so that most appropriate dosage form is accomplished taking into account of the administration route, bioavailability and adverse effect and may include sustained release formulations, topically applied formulations (lozenges, buccal formulations, sublingual formulations), drug controlled release formulations, enteric coated formulations and formulations soluble in stomach.

Components for drug delivery system essentially comprise a drug, a drug release module, a coating component and a therapy program. Then the drug of shorter half life is preferred, because it permits rapid decline of the blood concentration particularly upon cessation of the release thereof. The coating is preferably nonreactive to the active tissue of the site to which the drug is administered. Further, the program is preferably configured so that the most optimal drug concentration is kept during the predetermined period. Then the drug release module substantially has a drug storage, a release control part, an energy source, and a release opening or a release surface. All of these fundamental components are not necessarily and thus addition or deletion of any of them may be optionally performed according to the circumstance.

Examples of materials to be used for drug delivery system may include polymers, cyclodextrin derivatives and lecithin.

Available polymer may include insoluble polymers (silicone, ethylene-vinyl acetate copolymer, ethylene-vinyl alcohol copolymer, ethylcellulose and cellulose acetate), water soluble polymers and hydroxyl gel-forming polymers (polyacrylamide, polyhydroxyethyl methacrylate cross-linked form, polyacryl cross-linked form, polyvinyl alcohol, polyethyleneoxide, water soluble cellulose derivatives, cross-linked poloxamer, chitin and chitosan), slow dissolving polymers (ethyl cellulose, and a partial ester of methylvinyl ether-maleic anhydride copolymer), polymers soluble in stomach (hydroxylpropylmethyl cellulose, hydroxylpropyl cellulose, carmellose sodium, macrogol, polyvinylpyrrolidone, and dimethylaminoethyl methacrylate-methyl methacrylate copolymer), enteric polymers (hydroxylpropylmethyl cellulose phthalate, cellulose acetate phthalate, hydroxylpropylmethyl cellulose acetate succinate, carboxymethylethyl cellulose and acrylic acid polymers), biodegradable polymers (heat coagulation or cross-linked albumin, cross-linked gelatin, collagen, fibrin, polycyanoacrylate, polyglycolic acid, polylactic acid, poly O-hydroxyacetic acid and polycaprolactone), any of which can be selected appropriately based on the dosage form.

Of the foregoing polymers, silicone, ethylene-vinyl acetate copolymer, ethylene-vinyl alcohol copolymer and a partial ester of methylvinyl ether-maleic anhydride copolymer can be used for the control of drug release. Then, cellulose acetate can be used as a material of a osmotic pressure pump. Further, ethyl cellulose, hydroxypropylmethyl cellulose, hydroxypropyl cellulose, methyl cellulose can be used as a material of a membrane of slow dissolving formulations. Then, polyacryl cross-linked form can be used as an attaching agent to mucosa.

Further, the formulation can be manufactured by adding, as a component for drug delivery system, solvent, excipient, coating agent, base, binding agent, lubricant, disintegrant, solubilizing agent, suspending agent, thickening agent, emulsifying agent, stabilizing agent, buffering agent, isotonizing agent, soothing agent, preservative agent, flavoring agent, fragrance agent and coloring agent in compliance with their dosage form like for oral administration, injection and suppository.

Although illustrative examples on the foregoing additives are listed below, they would not be limited thereby.

[SOLVENT] purified water, water for injection, saline, peanut oil, ethanol, glycerol.

[EXCIPIENT] starches, lactose, glucose, sucrose, crystalline cellulose, calcium sulfate, calcium carbonate, talc, titanium oxide, trehalose, xylitol.

[COATING AGENT] sucrose, gelatin, cellulose acetate phthalate and the foregoing polymers.

[BASE] vaseline, vegetable oil, macrogol, base for oil in water emulsion, base for water in oil emulsion.

[BINDING AGENT] natural polymer compounds such as starch and derivatives thereof, cellulose and derivatives thereof, gelatin, sodium alginate, gum tragacanth and gum Arabic, synthetic polymers such as polyvinylpyrrolidone, dextrin, hydroxylpropyl starch.

[LUBRICANT] stearic acid and salts thereof, talc, waxes, wheat starch, macrogol, hydrogenated vegetable oil, sucrose fatty acid ester, polyethylene glycol.

[DISINTEGRANT] starch and derivatives thereof, agar, gelatin powder, sodium bicarbonate, cellulose and derivatives thereof, carmellose calcium, hydroxypropyl starch, carboxymethyl cellulose, and salts and derivatives thereof, poorly substituted hydroxypropyl cellulose.

[SULUBILIZING AGENT] cyclodextrin, ethanol, propylene glycol, polyethylene glycol.

[SUSPENDING AGENT] gum arabic, gum tragacanth, sodium alginate, aluminum monostearate, citric acid, various surfactants.

[THICKENING AGENT] carmellose sodium, polyvinylpyrrolidone, methyl cellulose, hydroxypropylmethyl cellulose, polyvinyl alcohol, gum tragacanth, gum arabic, sodium alginate.

[EMULSIFYING AGENT] gum arabic, cholesterol, gum tragacanth, methyl cellulose, various surfactants, lecithin.

[STABILIZING AGENT] sodium bisulfite, ascorbic acid, tocopherol, chelating agent, inert gas, reducing agent.

[BUFFERING AGENT] sodium hydrogenphosphate, sodium acetate, boric acid.

[ISOTONIZING AGENT] sodium chloride, glucose.

[SOOTHING AGENT] procaine hydrochloride, lidocaine, benzyl alcohol.

[PRESERVATIVE AGENT] benzoic acid and salts thereof, p-oxybenzoic esters, chlorobutanol, inverted soap, benzyl alcohol, phenol, thimerosal.

[FLAVORING AGENT] sucrose, saccharin, glycyrrhiza extract, sorbitol, xylitol, glycerin.

[FRAGRANCE AGENT] orange peel tincture, rose oil.

[COLORING AGENT] water soluble edible dye, lake dye.

The present invention will be described in more detail by the following non-limiting illustrative examples, but the present invention should not be construed to be limited by these examples.

### EXAMPLES

### Example 1

### Search on EST Database

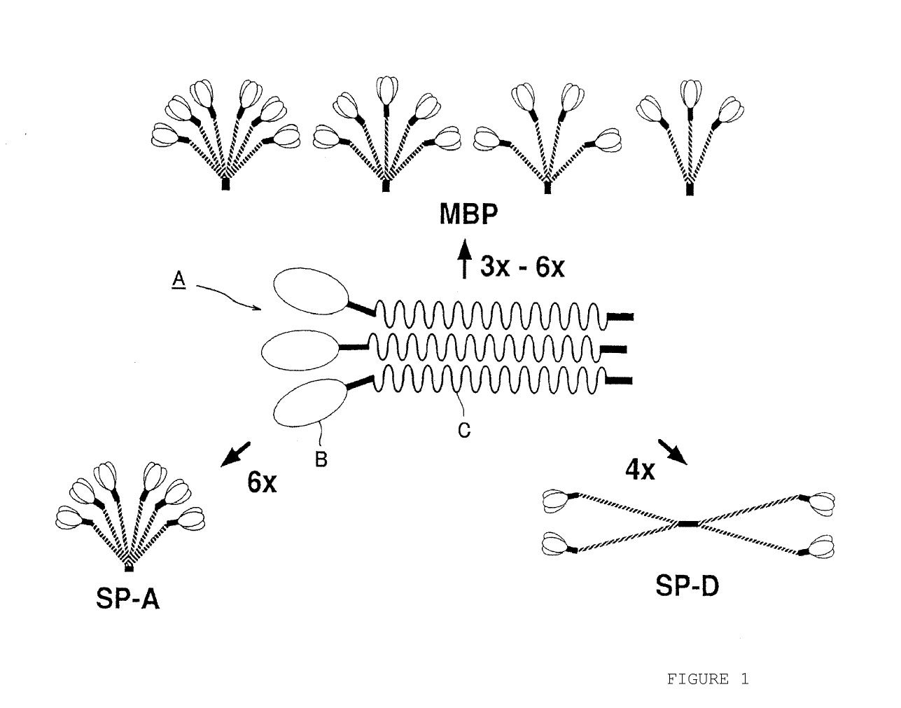

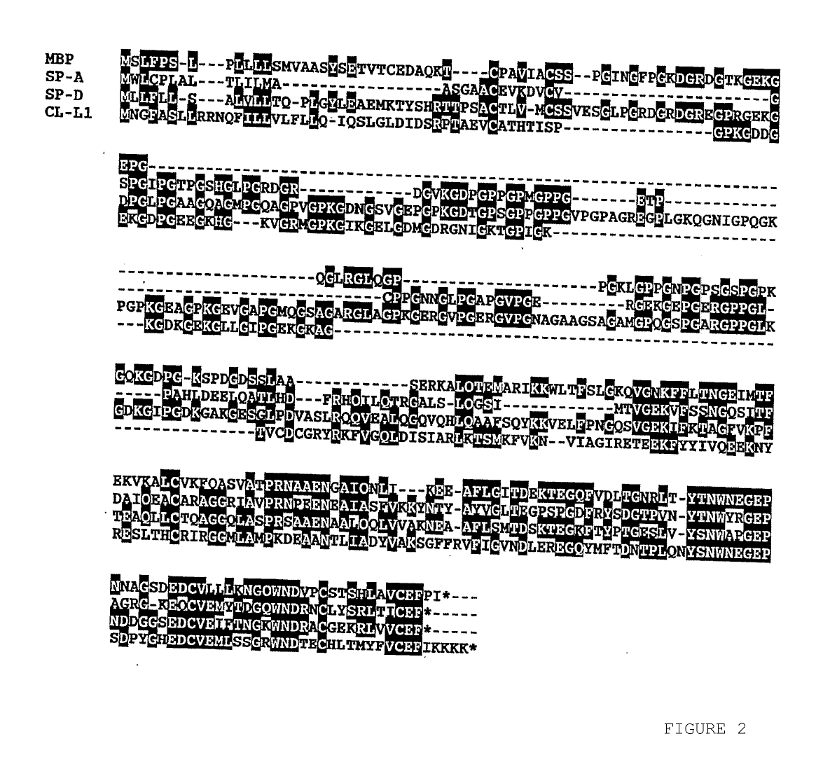

FIG. 2 illustrates homology of amino acid residues of known collectins (human MBP, human SP-A and human SP-D) as depicted in FIG. 1 and those of collectin CL-L1 derived from human liver which was successfully isolated recently by the present inventor (See, Japanese Patent Provisional Publication No. 11-206377). In FIG. 2, portions of amino acid residues that were recognized as being homologous were boxed.

An amino acid sequence of consecutive 159 amino acids (SEQ ID NO: 13) in carbohydrate chain recognition domain which is responsible for a lectin activity of CL-L1 was used to search on EST (Expressed Sequence Tags) database. As a result, several data containing a highly homologous amino acid sequence were searched.

Amino acid sequence information so obtained were searched on GenBank/EST database and were determined as to whether they were either of known or unknown substance. Consequently, one datum (H30455, derived from thymus) that exhibits high homology but contains an unknown base sequence could be obtained. Using base sequence of the EST clone thus obtained, search of EST database was again performed to take nine data (accession numbers: AA558494, derived from germ cells; AA582499, derived from kidney; AI420986, derived from prostate gland; AA742449, derived from germ cells; AA954657, derived from kidney; AA908360, derived from ovary; AI264145, derived from kidney; AA089855, derived from heart; AA456055, derived from melanocyte, pregnant uterus, fetal heart) that were found to include an identical base sequence.

All of these were clones which demonstrate a part of a base sequence of an identical collectin.

### Example 2

### Screening from a cDNA library derived from human liver by PCR and Sequencing of Nucleotide Sequence

A consensus sequence (SEQ ID NO: 14) was produced in view of nucleotide sequences of ten clones taken in Example 1. Then, in order to cloning upstream region (5′-direction) of the target human collectin cDNA, two primers toward the upstream direction, namely, CAP1 (5′-agattttattgtatagcttgg-3′ (SEQ ID NO: 15)) and CAP2 (5′-ctgggtaataattacataatg-3′ (SEQ ID NO: 16) were synthesized with 392A DNA/RNA synthesizer (PE Applied Biosystems Inc.,) based on the consensus sequence. Then, primes pertinent to a part of vector region in the cDNA library derived from human liver, namely, λTriplEx-F1 (5′-aagctccgagatctggacgag-3′ (SEQ ID NO: 17)) and λTriplEx-F2 (5′-ctcgggaagcgcgccattgtg-3′ (SEQ ID NO: 18)) were similarly synthesized.

Then, screening by polymerase chain reaction (PCR) was performed as described below (FIG. 3).

First PCR was performed using a cDNA library derived from human (Clontech Co.,) as a template. Reaction mixture solution in a total volume of 50 μL contained LA PCR Buffer II (magnesium ion free), 2.5 mM magnesium chloride, each 1 μL of 200 μM dATP, dCTP, dGTP and dTTP (any of which is manufactured by Takara Shuzo Co., Ltd.), cDNA library derived from human liver (Clontech Co.,), 0.5 M λTriplEx-F1 primer and 0.5 μM CAP1 primer.

PCR was performed with a program of 35 cycles consisting of heat denaturation at 95° C. for 20 seconds, annealing at 60° C. for seconds, elongation reaction at 72° C. for 90 seconds, in addition thereto, heat denaturation at 95° C. for 5 minutes prior to the repeated reaction, and final elongation reaction at 72° C. for 5 minutes.

After completing the first PCR, second PCR was performed.

1 μL PCR product in the first PCR was used as a template, and λTriplEx-F2 primer and CAP2 primer were used. Second PCR was performed according to a similar reaction constitution and program (except that the cycle number was 25 cycles) employed in the first PCR. Second PCR was performed with GeneAmp PCR System9700 (PE Applied Biosystems Inc.). Thus resulting PCR product was applied onto an agarose gel electrophoresis, and excised from the gel followed by freezing at −80° C. for 10 minutes. After centrifuging the frozen samples at 15,000 rpm for 10 minutes, the product was purified by ethanol precipitation of the supernatant.

Purified DNA fragment was incorporated into pT7Blue Vector (Novagen CO.,) and the vector was transformed into competent cells, XL1-Blue cells. The transformant was cultured in a LB medium (100 μg/mL ampicillin) followed by extraction of the plasmid with an alkaline SDS method to sequence the base sequence thereof with BigDye Terminator Cycle Sequencing FS Ready Reaction kit (PE Applied Biosystems Inc.) and ABI PRISM 377 sequencer (PE Applied Biosystems Inc.). Primers employed were M13 Universal Primer (5′-cgacgttgtaaaacgacggccagt-3′ (SEQ ID NO: 19)) and M13 Reverse Primer (5′-caggaaacagctatgac-3′ (SEQ ID NO: 20)). Both of them were synthesized similarly according to the methodology employed on CAP1 primer.

Nucleotide sequence so determined was slight difference from that of 5′-end region in the consensus sequence of EST obtained in Example 1, but it was clarified that the nucleotide sequence has additional 575 bases (a region corresponding to 68^{th}-271^{st} amino acids in hCL-K1 ORF denoted in FIG. 3) extending from 3′-end of CAP2 primer to N-terminal end thereof.

### Example 3

### Screening of hCL-K1 Polypeptide from a Cap Site cDNA Library Derived from Human Kidney and Sequencing of Nucleotide sequence

In order to cloning 5′-end region containing a transcription initiation site and nucleotide sequence taken in Example 2, two primers toward the upstream direction, namely, CAP3 (5′-ggtcctatgtcaccggaatc-3′ (SEQ ID NO: 21)), CAP4 (5′-ttccatgacgacccacactgc-3′ (SEQ ID NO: 22)) were synthesized with 392A DNA/RNA synthesizer (PE Applied Biosystems Inc.,)

Then, screening by polymerase chain reaction (PCR) was performed using a cap site cDNA according to the following steps (FIG. 3).

First PCR was performed with Cap Site cDNA, Human Kidney (NIPPON GENE Co., Ltd.) using attached 1RC2 primer (5′-caaggtacgccacagcgtatg-3′ (SEQ ID NO: 23)) and CAP3 primer.

Reaction mixture solution in a total volume of 50 μL contained LA PCR Buffer II (magnesium free), 2.5 mM magnesium cjloride, each 1 μL of 200 μM dATP, dCTP, dGTP and dTTP (any of which is manufactured by Takara Shuzo Co., Ltd.), a Cap Site cDNA Human kidney, 0.5 μM RC2 primer (NIPPON GENE Co., Ltd.), and 0.5 μM CAP3 primer. PCR was performed with a program of 35 cycles consisting of heat denaturation at 95° C. for 20 seconds, annealing at 60° C. for 20 seconds, elongation reaction at 72° C. for 60 seconds, in addition thereto, heat denaturation at 95° C. for 5 minutes prior to the repeated reaction, and final elongation reaction at 72° C. for 10 minutes.

After completing the first PCR, second PCR was performed. 1/L PCR product of the first PCR was used as a template, and attached 2RC2 primer (5′-gtacgccacagcgtatgatgc-3′ (SEQ ID NO: 24)) and CAP4 primer were used. Second PCR was performed according to a similar reaction constitution and program (except that the cycle number was 25 cycles) employed in the first PCR.

Second PCR was performed with GeneAmp PCR System9700 (PE Applied Biosystems Inc.). Thus resulting PCR product was applied onto an agarose gel electrophoresis, and excised from the gel followed by freezing at −80° C. for 10 minutes. After centrifuging the frozen samples at 15,000 rpm for 10 minutes, the product was purified by ethanol precipitation of the supernatant.

Purified DNA fragment was incorporated into pT7Blue Vector (Novagen CO.,) and the vector was transformed into competent cells, XL1-Blue cells. The transformant was cultured in a LB medium (100 μg/mL ampicillin) followed by extraction of the plasmid with an alkaline SDS method to sequence the base sequence thereof with BigDye Terminator Cycle Sequencing FS Ready Reaction kit (PE Applied Biosystems Inc.) and ABI PRISM 377 sequencer (PE Applied Biosystems Inc.). Primers employed were the foregoing M13 Universal Primer and M13 Reverse Primer.

It was clarified that the base sequence so determined was a nucleotide sequence that has additional 492 nucleotides extending from N-terminal end of the base sequence determined in Example 2. Namely, hCL-K1 polypeptide taken in this Example has open reading frame (ORF) consisting of consecutive 813 nucleotides set out in SEQ ID NO:1 which encodes consecutive 271 amino acids set out in SEQ ID NO:2.

### Example 4

### Homology Search

Homology search was conducted for DNA and amino acid on GenBank database. As a result, the amino acid sequence set out in SEQ ID NO:2 was not identical to any amino acid sequence of the known collectins and was therefore demonstrated that such amino acid sequence was novel.

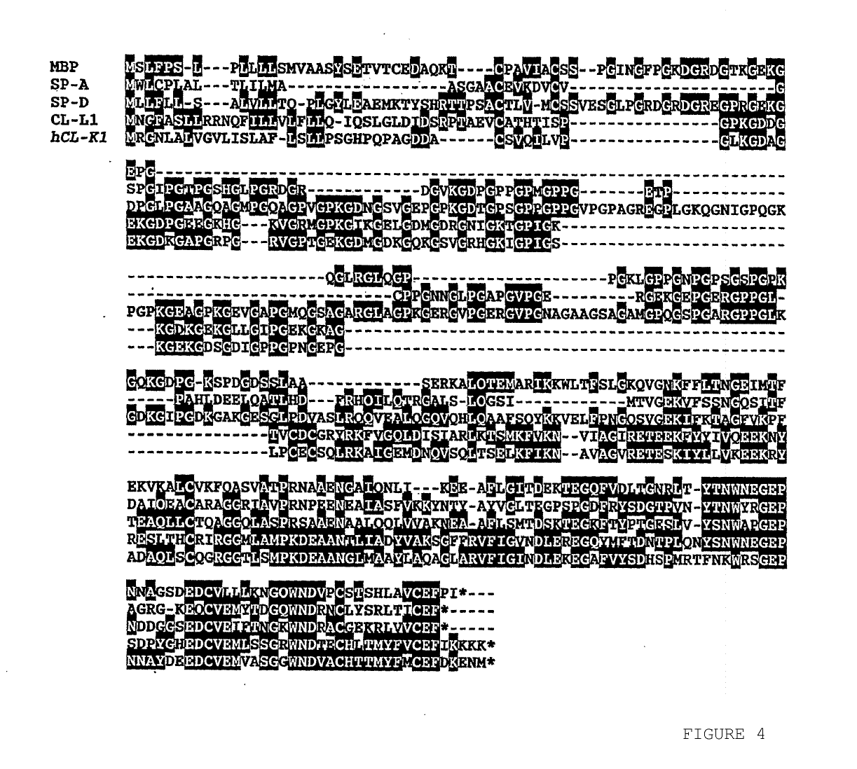

Amino acid residues of known collectins (human MBP, human SP-A and human SP-D) as depicted in FIG. 1 and those of collectin CL-L1 derived from human liver (See, Japanese Patent Provisional Publication No. 11-206377) was compared with the amino acid sequence of hCL-K1 polypeptide of the present invention. Result was shown in FIG. 4. Portions of amino acid residues that are recognized as being homologous were boxed.

According to this alignment, it was demonstrated that hCL-K1 polypeptide of the present invention has homology to known collectin, and that it belongs to a collectin family.

### Example 5

### Analysis of Expression Distribution of hCL-K1 Polypeptide in Human Tissues

In order to examine the expression of hCL-K1 polypeptide (SEQ ID NO:2) in various human tissues, analysis was performed by Reverse Transcriptase (RT)-PCR.

The following primers were prepared for RT-PCR. Namely, RTF1 (5′-agattccggtgacataggacc-3′ (SEQ ID NO: 25)) and RTR1 (5′-tggtctgggctctgtccctgc-3′ (SEQ ID NO: 26)) for amplifying cDNA sequence of from neck region to carbohydrate recognition domain in hCL-K1 polypeptide. Then, the other two primers were also prepared. Namely, human 8-actin sense primer (5′-caagagatggccacggctgct-3′ (SEQ ID NO: 27)) and human β-actin antisense primer (5′‘-tccttctgcatcctgtcggca-3′ (SEQ ID NO: 28)) for amplifying a part of β-actin gene for use in comparison of the amount of expressed hCL-K polypeptide in each of human tissues. All of these primers were synthesized in a similar manner to CAP1 primer.

Template was RNA from human tissues, namely, brain, heart, kidney, liver, lung, trachea, bone marrow, colon, small intestine, spleen, stomach, thymus, mammary gland, prostate gland, skeletal muscle, testis, uterus, cerebellum, fetal brain, fetal liver, spinal cord, placenta, adrenal gland, pancreas, salivary gland and thyroid. Then RT-PCR was performed using RNA LA PCR Kit (AMV) Ver.1.1 (TAKARA Syuzo, Co.).

First of all, Reverse Transcriptase reaction was performed according to the following reaction scheme.

5 mM magnesium chloride, 1×RNA PCR Buffer, 1 mM dNTP Mixture, 1 U/μl RNase inhibitor and 2 μg of RNA were mixed, and total volume of the mixture was adjusted to realize 401 with RNase free distilled water. At the same time, a reaction mixture without reverse transcriptase was also prepared for a negative control. These reaction mixture solution was placed in 0.2 ml tube, and subjected to a reverse transcription reaction with GeneAmp PCR System9700 (PE Applied Biosystems Inc.) through 1 cycle consisting of 30 minutes at 42° C., 5 minutes at 99° C. and 5 minutes at 5° C. 10 μL of the resulting reverse transcription reaction product was subsequently used for LA PCR in the following reaction mixture with 28 cycles and 35 cycles respectively. 2.5 mM magnesium chloride, 1×LA PCR Buffer (magnesium ion free), 2U TaKaRa LA Taq, 0.2 μM RTF1 primer and 0.2 M RTR1 primer were mixed, and the mixture was adjusted to realize total volume of 50 μL with sterilized distilled water. PCR was performed with a program of 28 cycles or 35 cycles consisting of heat denaturation at 95° C. for 20 seconds, annealing at 60° C. for 20 seconds and elongation reaction at 72° C. for 60 seconds, in addition thereto, heat denaturation at 95° C. for 5 minutes prior to the repeated reaction and final elongation reaction at 72° C. for 10 minutes.

The reaction product was separated by 1.5% agarose gel electrophoresis, followed by staining with ethidium bromide solution (0.1 μg/mL), confirmation of the electrophoretic pattern with transilluminator, and identification of any expression tissue. In order to compare an amount of expression in each of the tissues, RT-PCR was performed to amplify a part of β-actin with each of the tissues, and RNA was corrected accordingly. With regard to β-actin, the serial steps of reverse transcriptase reaction, PCR and judgment had also been performed.

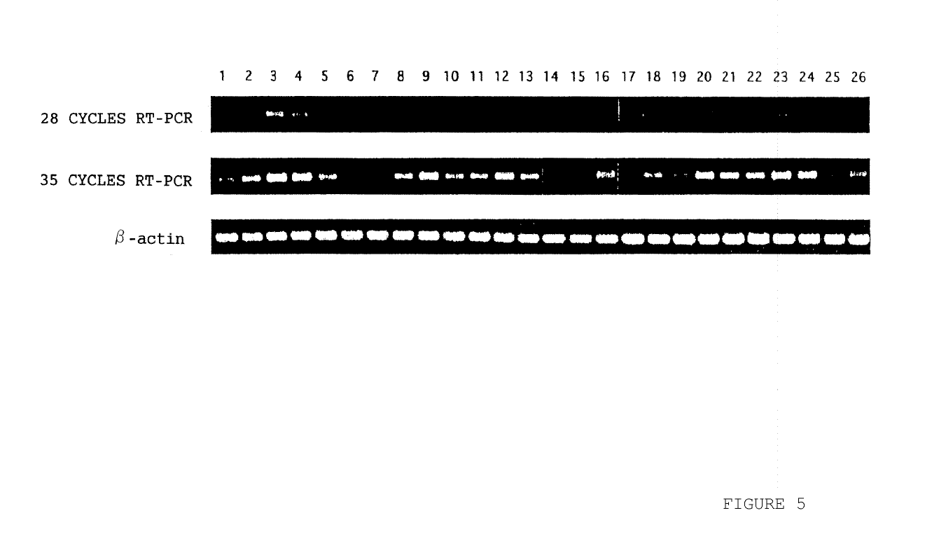

Results were shown in FIG. 5. Tissue (Lane Number) allocated to each lane in FIG. 5 is as follows. Brain (1), Heart (2), Kidney (3), Liver (4), Lung (5), Trachea (6), Bone Marrow (7), Colon (8), Small Intestine (9), Spleen (10), Stomach (11), Thymus (12), Mammary Gland (13), Prostate Gland (14), Skeletal Muscle (15), Testis (16), Uterus (17), Cerebellum (18), Fetal Brain (19), Fetal Liver (20), Spinal Cord (21), Placenta (22), Adrenal Gland (23), Pancreas (24), Salivary Gland (25) and thyroid (26).

There were expression of hCL-K1 polypeptide, according to PCR performed with 28 cycles, in Kidney (3) intensively, and also in Liver (4), Small Intestine (9), Thymus (12), Fetal Liver (20), Spinal Cord (21), Adrenal Gland (23) and Pancreas (24). Further, with respect to PCR performed with 35 cycles, ubiquitous expression of hCL-K1 polypeptide could be confirmed in all tissues tested though some difference on expression level has been observed therein.

### Example 6

### Genetic Analysis of hCL-K1 Polypeptide

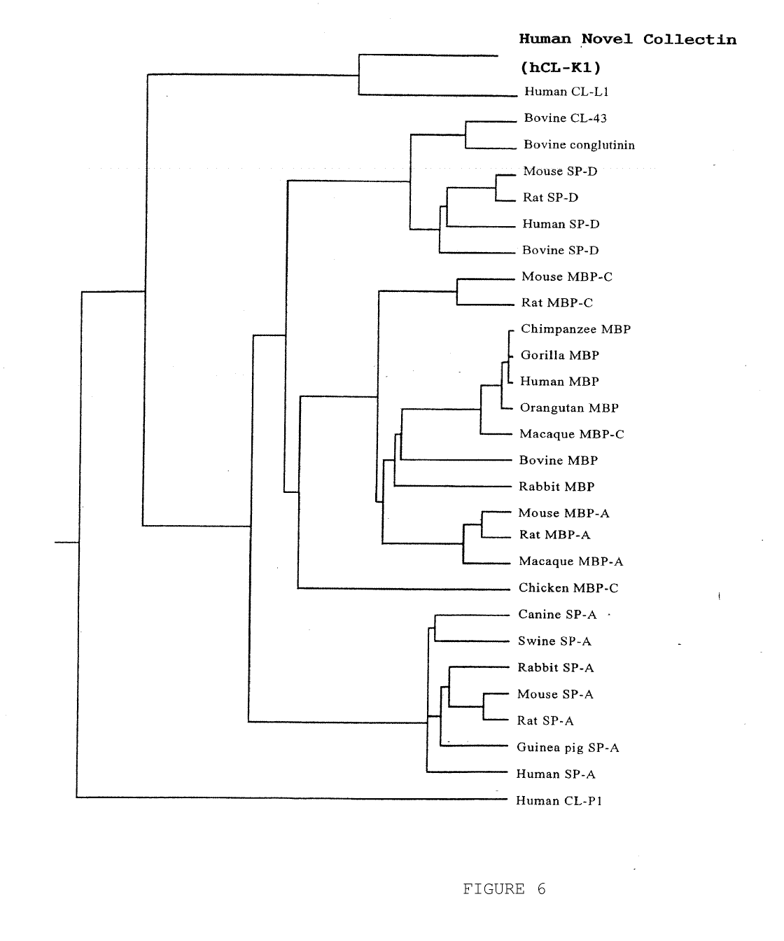

By comparing hCL-K1 polypeptide with known collectins, an analysis had been conducted to prepare a phylogenetic tree for clarifying genetic position of it thereamong. Collectins subjected to such analysis were shown in FIG. 6. In FIG. 6, polypeptides of CL-L1 and C1-P1 were successfully isolated by the present inventors (supra).

Multiple alignment was prepared with clustalw method using a region containing a lectin domain based on data obtained by searching each amino acid sequence in GenBank database.

A phylogenetic tree was prepared with Phylip version 3.57c package program using N-J process (neighbor-joining process) based on the foregoing multiple alignment. Consequently, it was revealed that SP-D, bovine CL-43 and bovine conglutinin formed single cluster, whilst MBP and SP-A respectively form separate clusters. In contrast thereto, hCL-K1 polypeptide did not belong in any of these clusters but in the same cluster which is similar to CL-L1. Accordingly, it was speculated that hCL-K1 polypeptide is a homologue of CL-L1.

### Example 7

### Analysis of Expression Distribution of hCL-K1 Polynucleotide in Human Tossues

In order to examine expression of hCL-K1 polynucleotide (SEQ ID NO:1) in various human tissues, an analysis with RT-PCR technique was performed.

The following primers were prepared. Namely, RTF2 (5′-atgagggggaatctggccctggtg-3′ (SEQ ID NO: 29)) and RTR2 (5′-catgttctccttgtcaaactcac-3′ (SEQ ID NO: 30)) for amplifying whole translation region of hCL-K1 polynucleotide. Then, the other two human β-actin primers noted in Example 5 were also prepared for amplifying a part of β-actin gene for use in comparison of the expression amount.

All of these primers were synthesized in a similar manner to CAP1 primer.

Then, RT-PCR was performed similarly to Example 5.

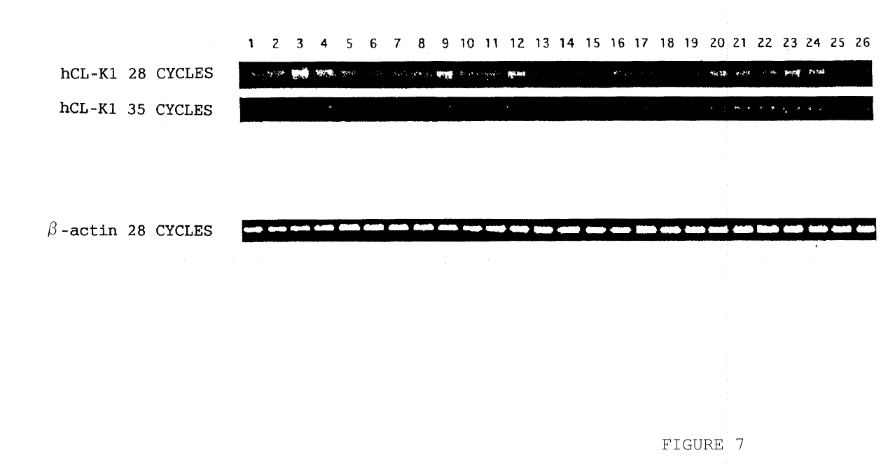

Results were shown in FIG. 7. Tissue (Lane Number) allocated to each lane in FIG. 7 is as follows. Brain (1), Heart (2), Kidney (3), Liver (4), Lung (5), Trachea (6), Bone Marrow (7), Colon (8), Small Intestine (9), Spleen (10), Stomach (11), Thymus (12), Mammary Gland (13), Prostate Gland (14), Skeletal Muscle (15), Testis (16), Uterus (17), Cerebellum (18), Fetal Brain (19), Fetal Liver (20), Spinal Cord (21), Placenta (22), Adrenal Gland (23), Pancreas (24), Salivary Gland (25) and thyroid (26).

There were intensive expression of hCL-K1 polynucleotide, according to PCR performed with 28 cycles, in Kidney (3), Liver (4), Small Intestine (9), Thymus (12), Fetal Liver (20), Spinal Cord (21), Adrenal Gland (23) and Pancreas (24). Further, with respect to PCR performed with 35 cycles, ubiquitous expression of hCL-K1 polynucleotide (SEQ ID NO:1) could be confirmed in all tissues tested though some difference on expression level has been observed therein.

Further, several amplified fragments were found in RT-PCR products of hCL-K1 polynucleotide as shown in FIG. 7. These bands were excised from the gel, and DNA fragments were purified by a similar process to that in Example 2, i.e., by freezing at −80° C. for 10 minutes, centrifuging at 15,000 rpm for 10 minutes followed by ethanol precipitation of the supernatant. Purified DNA fragments were incorporated into pT7Blue Vector (Novagen Co.), and thus resulting vector was transformed into competent cells, XL1-Blue cells. Transformant was cultured in a LB medium (100 μg/ml ampicillin), and then a plasmid was extracted by an alkali SDS method. Base sequence was determined with BigDye Terminator Cycle Sequencing FS Ready Reaction kit (PE Applied Biosystems Inc.) and ABI PRISM 377 sequencer (PE Applied Biosystems Inc.). Primers employed were M13 Universal Primer (5′-cgacgttgtaaaacgacggccagt-3′ (SEQ ID NO: 19)) and M13 Reverse Primer (5′-caggaaacagctatgac-3′ (SEQ ID NO: 20)).

Due to alternative splicing of mRNA, hCL-K1 polypeptide (SEQ ID NO:2) had three mutant polypeptides of hCL-K1v1 (SEQ ID NO:5), hCL-K1v2 (SEQ ID NO:8) and hCL-K1v3 (SEQ ID NO:11).

hCL-K1v1 polypeptide does not have 44^{th}-91^{st} amino acids in SEQ ID NO: 2 (394^{th}-537^{th} nucleotides in SEQ ID NO: 1) and amino acid sequence thereof is encoded by the nucleotide sequence set out in SEQ ID NO: 6.

hCL-K1v2 polypeptide does not have 44^{th}-67^{th} amino acids in

SEQ ID NO: 2 (394^{th}-465^{th} nucleotides in SEQ ID NO: 1) and amino acid sequence thereof is encoded by the nucleotide sequence set out in SEQ ID NO: 9.

hCL-K1v3 polypeptide does not have 68^{th}-91^{st} amino acids in

SEQ ID NO: 2 (466^{th}-537^{th} nucleotides in SEQ ID NO: 1) and amino acid sequence thereof is encoded by the nucleotide sequence set out in SEQ ID NO: 12.

### Example 8

### Construction of Expression Vector Incorporating hCL-K1 Polynucleotide

Translated region of hCL-K1 polynucleotide (SEQ ID NO: 1) was amplified, with a cDNA library derived from human kidney as a template, by PCR (Takara Thermal Cycler MP; Takara Shuzo Co., Ltd.) which employs CL-L2-1F primer (5′-gggaagcttcgatcaggatgagggggaatctggccctggtg-3′ (SEQ ID NO: 31)) and CL-L2-1R primer (5′-gggctcgagcatgttctccttgtcaaactcac-3′ (SEQ ID NO: 32)). Resulting hCL-K1 polynucleotide was ligated to pT7Blue T-Vector (Novagen Co.) and was transformed into Escherichia coli XLI-Blue.

A plasmid containing hCL-K1 polynucleotide was purified from the resulting clone. Base sequence of the resulting plasmid was confirmed with a sequencer, then the plasmid with no error was digested with restriction enzymes Hind III and Xho I, and was ligated to pcDNA3.1/Myc-His(+)A vector (Invitrogen Co,.) that had been digested with the same enzymes and had been purified.

Ligated plasmid was transformed into Escherichia coli XLI-Blue, the resulting clone was cultured. The plasmid was then purified to realize an expression vector pcDNA3.1/Myc-His(+)A-CL-L2-1.

At the same instant, expression vectors were similarly produced for mutant of hCL-K1 polypeptide, namely, hCL-K1v1, hCL-K1v2 and hCL-K1v3.

### Example 9

### Production of Cell Strain which Stably Expresses hCL-K1 Polypeptide

Transient expression was performed through cotransfection of the expression vector pcDNA3.1/Myc-His(+)A-CL-L2-1 obtained in Example 8 and pEGFP-F vector (Clontech Co.,) into CHO cells using LIPOFECTAMINE 2000 (LF2000) Reagent (GIBCO BRL Co.).

0.5 ml solution of LF2000 Reagent (LF2000 Reagent 301, Nutrient Mixture F-12 Ham (Ham's F-12 medium; Sigma Co.)) was first prepared, and was incubated at room temperature for 5-minutes. Then, 0.5 ml of a vector solution (pcDNA3.1/Myc-His(+)A-CL-L2-1: 7.5 μg, pEGFP-F vector 2.5 μg, Ham's F-12 medium) was admixed therewith, followed by incubation for 20 minutes. Thereafter, the solution was added to CHO cells that had been cultured to a high density in a 25 cm² flask including 5 ml of Ham's F-12 medium (containing 5% FCS). After incubation at 37° C. for 4 hours in the presence of 5% CO₂, the medium was replaced with a flesh medium, followed by subsequent incubation at 37° C. for 20 hours in the presence of 5% CO₂. Next, the medium was replaced with Ham's F-12 medium (containing 5% FCS, 0.4 mg/ml Geneticin (GIBCO BRL Co.)), and 10 days culture was subsequently performed. In this process, the medium was once replaced.

In this 10 day drug selection, transformed cells could be survived and be proliferated, however, non-transformed cells were died. In order to obtain highly expressing cells from the resulting transformed cells, sorting was performed by a cell sorter (Becton Dickinson Co.) with fluorescence of GFP as a marker. After washing twice the transformed cells in the 25 cm² flask with 5 ml PBS(−), the cells were stripped off with 0.3 ml of 0.02% EDTA solution (Nakarai Tesc KK). The cells were suspended in 10 ml PBS(−), and were centrifuged at 200×g for 7 minutes at 4° C. to remove the supernatant. The remaining cells were suspended in 0.5 ml of 2% FCS/PBS(−) to realize a sorting sample.

After the sample was passed through a 5 ml tube (Becton Dickinson Co.) equipped with a cell strainer cap, it was applied to a cell sorter. Non-transformed CHO cells treated similarly were used as control cells. Selected cells were those which exhibited 10 times or more fluorescence intensity than the control cells. These cells were dispensed into 96-well cell culture plates contained 100 μl Ham's F-12 medium (containing 5% FCS, 0.4 mg/ml Geneticin) to put a single cell per well. After the cells were cultured at 37° C. in the presence of 5% CO₂ for one week, each 100 μl of the culture medium was further added thereto followed by the additional culture for one week. A clone proliferated by drug selection with Geneticin was divided into two parts, and then they were transferred to 12-well and 24-well cell culture plates respectively. At that time, some clones proliferated from two or more cells in the single well were excluded, and the other cells were plated onto 12-well and 24-well cell culture plates at a cell number ratio of 9:1.

Cells were cultured at 37° C. in the presence of 5% CO₂ until the cells in the 12-well plate reach to high density. Then 200 μl of the culture supernatant was dot blotted on an Immobilon-P membrane (Millipore Co., Ltd.) using Bio-Dot Microfiltration Apparatus (BIO-RAD Co., Ltd.).

Further, the membrane was incubated in a solution of anti-myc antibody (Invitorogen Co.,) diluted 5,000 times in 0.05% Tween 20/TBS buffer (Takara Shuzo Co., Ltd.) at room temperature for 1 hour. Then the membrane was washed three times with 100 ml of 0.05% Tween 20/TBS buffer at room temperature for 20 minutes, followed by further incubation in a solution of anti-IgG-HRP (Chemicon Co., Ltd.) diluted 5,000 times in 0.05% Tween 20/TBS buffer at room temperature for 1 hour. Thereafter, the membrane was washed three times with 100 ml of 0.05% Tween 20/TBS buffer at room temperature for 20 minutes, followed by detection using TMB Membrane Peroxidase substrate system (Funakoshi KK). After confirming the clones with intense color development, cells in each corresponding well of the 24-well plate were identified as a stably expressing cell strain (CHO/CL-K1).

### Example 10

### Analysis on Sugar Binding Specificity of hCL-K1 Polypeptide

One litter of the culture supernatant of the stably expressing cell strain (CHO/CL-K1) produced in Example 9 was concentrated to 50 ml using VIVAPORE10 (Funakoshi KK), thereafter was added 200 μl of Ni-NTA agarose (Quiagen Co., Ltd.) thereto.

hCL-K1 polypeptide was bound to mannan agarose by incubation of the mixture with shaking at 4° C. overnight. Mannan agarose was packed in Poly-Prep Chromatography Columns (BIO-RAD Co., Ltd.) followed by washes three times with 5 ml of 50 mM NaH₂ PO₄, 300 mM NaCl, 20 mM imidazole, 0.05% Tween20 (pH8.0), and by elution five times with 200 μl of 50 mM NaH₂ PO₄, 300 mM NaCl, 250 mM imidazole, 0.05% Tween20 (pH8.0) to purify hCL-K1 polypeptide. Purified hCL-K1 polypeptide was quantitatively determined, and used for the analysis of sugar specificity.

Namely, this Example had examined binding activity for the various sugar agarose (mannan, mannose, fucose, N-acetylglucosamine, maltose, N-acetylgalactosamine) to be offered by hCL-K1 polypeptide and MBL respectively purified with mannan agarose. 200 μl of 50% sugar agarose/TBSC (Tris Buffer, 5 mM calcium chloride) was added to one ml of 4 μg/ml hCL-K1 polypeptide and MBL, and they were incubated with gentle agitation for 12 hours at 4° C. Then, they were centrifuged for minutes at 3,000 rpm, supernatant so produced were collected and their agarose were washed with TBSC. Elution was performed with 1 ml of TBSE (Tris Buffer, 10 mM EDTA), then they were subjected to western blotting.

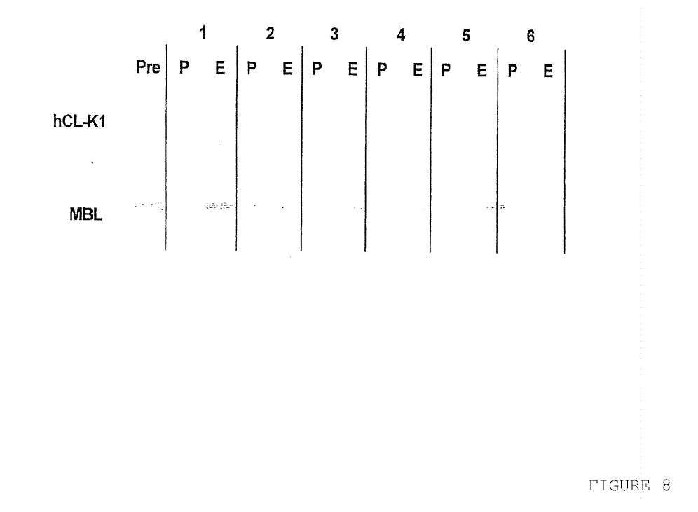

Results were shown in FIG. 8. Sugar agarose (Lane Number) allocated to each lane in FIG. 8 is as follows. Mannan (1), Mannose (2), Fucose (3), N-acetylglucosamine (4), maltose (5) and N-acetylglucosamine (6). Then the treatment schemes employed in each of lanes illustrated in FIG. 8 are non-purified with agarose (Pre), fraction without bound agarose (P) and fraction eluted with TBSE (E).

Apparently from the results shown in FIG. 8, hCL-K1 polypeptide bound to mannan, fucose and N-acetylglucosamine. But hCL-K1 polypeptide did not bind to maltose and N-acetylglucosamine. This clearly indicated that although both hCL-K1 polypeptide and MBL have mannose binding lectin domain, their specific binding activities to the sugars are not identical.

### Example 11

### Separation of hCL-K1 Polypeptide from Serum

By making use of the unique specific binding activity in hCL-K1 polypeptide demonstrated in Example 10, hCL-K1 polypeptide was purified from serum.

Namely, fucose agarose was added to serum, and they were incubated with gentle agitation for 12 hours at 4° C. Agarose was then washed with TBSC. Elution was performed with TBSE (Tris Buffer, 10 mM EDTA), then calcium chloride up to final concentration of 20 mM was added to maltose agarose. They were incubated with gentle agitation, and agarose was washed with TBSC. Further, elution was performed with TBSE (Tris Buffer, 10 mM EDTA), then they were subjected to western blotting.

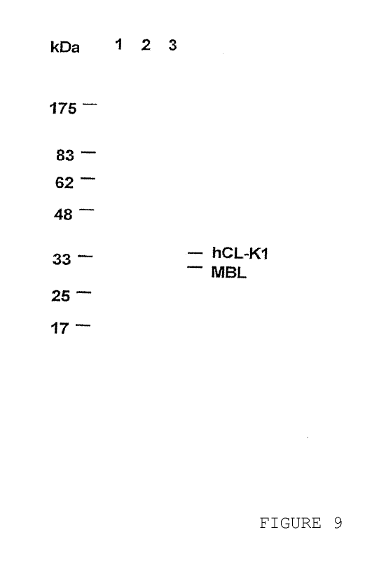

Results were shown in FIG. 9. Sample (Lane Number) allocated to each lane in FIG. 9 is as follows. Fraction wherein serum was added to fucose aragose and eluted with TBSE (1), Fraction without bound maltose (2) and Fraction eluted with TBSE from maltose agarose (3).

In the fraction eluted from fucose agarose, both hCL-K1 polypeptide and MBL were eluted, on the other hand, only MBP bound to maltose agarose and was eluted therefrom though hCL-K1 polypeptide did not bind thereto.

In consideration of their polypeptide structure, it was expected that both substances might have similar specific binding activities to the sugars, but the results indicated incompatible activities. Such unique specific binding activity offered by hCL-K1 polypeptide can not easily be predictable from their amino acid sequence (structure) and was firstly demonstrated through the research by the present inventors. Based on such specific binding activity, hCL-K1 polypeptide will be used as a tool to separate it from the other lectins.

### INDUSTRIAL APPLICABILITY

hCL-K1 polypeptide of the present invention is effectively available for the elucidation of mechanisms in a wide variety of diseases such as bacterial infections which involves with human immune function, and for the development of reagents and drugs for the diagnosis, prophylaxis and therapy on such diseases.

1-25. (canceled)

26. An expression vector pcDNA3.1/Myc-His(+)A-CL-L2-1.

27. A method for producing a polypeptide comprising the steps of:
(a) transforming a host cell with the vector according to claim 1,
(b) culturing the host cell in a culture medium, and
(c) harvesting the polypeptide produced by the host cell.

28. The method according to claim 2 wherein said polypeptide consists of 271 consecutive amino acids set out in SEQ ID NO:2, and does not bind to both maltose and N-acetylgalactosamine.

29. The method according to claim 2 or 3 wherein said polypeptide is encoded by a polynucleotide which consists of 813 consecutive nucleotides set out in SEQ ID NO:3.

30. The method according to claim 2 wherein said host cell is an animal cell.


SEQUENCE LISTING (32 sequences)

SEQ ID NO: 1 - DNA, 1341 bp, Homo sapiens
  CDS (265)..(1077)
cgcggccgcgtcgacggacggtggacgcagcgcagacaggaagctccccgagataacgctgcggccgggcggcctgatttgctgggctgtctgatggcccgggccgaggcttctccctgcgcctgggactgcggccgcctctctaaatagcagccatgaggcgcctgggggcagtgtcctcgcggccgcgtcgaccgacggccgcagtcgacgccccgttcgcctagcgcgtgctcaggagttggtgtcctgcctgcgctcaggatgagggggaatctggccctggtgggcgttctaatcagcctggccttcctgtcactgctgccatctggacatcctcagccggctggcgatgacgcctgctctgtgcagatcctcgtccctggcctcaaaggggatgcgggagagaagggagacaaaggcgcccccggacggcctggaagagtcggccccacgggagaaaaaggagacatgggggacaaaggacagaaaggcagtgtgggtcgtcatggaaaaattggtcccattggctctaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatgtgagcctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccag

SEQ ID NO: 2 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 3 - DNA, 813 bp, Homo sapiens
atgagggggaatctggccctggtgggcgttctaatcagcctggccttcctgtcactgctgccatctggacatcctcagccggctggcgatgacgcctgctctgtgcagatcctcgtccctggcctcaaaggggatgcgggagagaagggagacaaaggcgcccccggacggcctggaagagtcggccccacgggagaaaaaggagacatgggggacaaaggacagaaaggcagtgtgggtcgtcatggaaaaattggtcccattggctctaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatg

SEQ ID NO: 4 - DNA, 1197 bp, Homo sapiens
  CDS (265)..(933)
cgcggccgcgtcgacggacggtggacgcagcgcagacaggaagctccccgagataacgctgcggccgggcggcctgatttgctgggctgtctgatggcccgggccgaggcttctccctgcgcctgggactgcggccgcctctctaaatagcagccatgaggcgcctgggggcagtgtcctcgcggccgcgtcgaccgacggccgcagtcgacgccccgttcgcctagcgcgtgctcaggagttggtgtcctgcctgcgctcaggatgagggggaatctggccctggtgggcgttctaatcagcctggccttcctgtcactgctgccatctggacatcctcagccggctggcgatgacgcctgctctgtgcagatcctcgtccctggcctcaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatgtgagcctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccag

SEQ ID NO: 5 - protein, 223 aa, Homo sapiens
MRGNLALVGVLISLAFLSLLPSGHPQPAGDDACSVQILVPGLKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 6 - DNA, 669 bp, Homo sapiens
atgagggggaatctggccctggtgggcgttctaatcagcctggccttcctgtcactgctgccatctggacatcctcagccggctggcgatgacgcctgctctgtgcagatcctcgtccctggcctcaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatg

SEQ ID NO: 7 - DNA, 1269 bp, Homo sapiens
  CDS (265)..(1005)
cgcggccgcgtcgacggacggtggacgcagcgcagacaggaagctccccgagataacgctgcggccgggcggcctgatttgctgggctgtctgatggcccgggccgaggcttctccctgcgcctgggactgcggccgcctctctaaatagcagccatgaggcgcctgggggcagtgtcctcgcggccgcgtcgaccgacggccgcagtcgacgccccgttcgcctagcgcgtgctcaggagttggtgtcctgcctgcgctcaggatgagggggaatctggccctggtgggcgttctaatcagcctggccttcctgtcactgctgccatctggacatcctcagccggctggcgatgacgcctgctctgtgcagatcctcgtccctggcctcaaaggagacatgggggacaaaggacagaaaggcagtgtgggtcgtcatggaaaaattggtcccattggctctaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatgtgagcctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccag

SEQ ID NO: 8 - protein, 247 aa, Homo sapiens
MRGNLALVGVLISLAFLSLLPSGHPQPAGDDACSVQILVPGLKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 9 - DNA, 741 bp, Homo sapiens
atgagggggaatctggccctggtgggcgttctaatcagcctggccttcctgtcactgctgccatctggacatcctcagccggctggcgatgacgcctgctctgtgcagatcctcgtccctggcctcaaaggagacatgggggacaaaggacagaaaggcagtgtgggtcgtcatggaaaaattggtcccattggctctaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatg

SEQ ID NO: 10 - DNA, 1269 bp, Homo sapiens
  CDS (265)..(1005)
cgcggccgcgtcgacggacggtggacgcagcgcagacaggaagctccccgagataacgctgcggccgggcggcctgatttgctgggctgtctgatggcccgggccgaggcttctccctgcgcctgggactgcggccgcctctctaaatagcagccatgaggcgcctgggggcagtgtcctcgcggccgcgtcgaccgacggccgcagtcgacgccccgttcgcctagcgcgtgctcaggagttggtgtcctgcctgcgctcaggatgagggggaatctggccctggtgggcgttctaatcagcctggccttcctgtcactgctgccatctggacatcctcagccggctggcgatgacgcctgctctgtgcagatcctcgtccctggcctcaaaggggatgcgggagagaagggagacaaaggcgcccccggacggcctggaagagtcggccccacgggagaaaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatgtgagcctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccag

SEQ ID NO: 11 - protein, 247 aa, Homo sapiens
MRGNLALVGVLISLAFLSLLPSGHPQPAGDDACSVQILVPGLKGDAGEKGDKGAPGRPGRVGPTGEKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 12 - DNA, 741 bp, Homo sapiens
atgagggggaatctggccctggtgggcgttctaatcagcctggccttcctgtcactgctgccatctggacatcctcagccggctggcgatgacgcctgctctgtgcagatcctcgtccctggcctcaaaggggatgcgggagagaagggagacaaaggcgcccccggacggcctggaagagtcggccccacgggagaaaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatg

SEQ ID NO: 13 - protein, 159 aa, Homo sapiens
CDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 14 - DNA, 619 bp, Artificial Sequence
  Consensus sequence
ttctcagatgattcctgtgcatggcgaagtttggaaactctgagtgttgcttttgagatcagacatacagtgctatccattggtgccctcgtggaggatggaacttacttgttgactnntgccacaccaccatgtacttcatgtgtgagtttgacaaggagaacatgtgaccctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccagaattgctcttccataaagcttgtgcctttgtccaagctatacaataaaatctttaagtagtgcagtagttaagtccaaaaagtggcaatggggtcttgaaaaaaaaaaaaaaaatttataaaaaaaaaagaactcactttgaccaacacttctgtaaattacattacaatataggttccttcacacta

SEQ ID NO: 15 - DNA, 21 bp, Artificial Sequence
  Primer CAP1
agattttattgtatagcttgg

SEQ ID NO: 16 - DNA, 21 bp, Artificial Sequence
  Primer CAP2
ctgggtaataattacataatg

SEQ ID NO: 17 - DNA, 21 bp, Artificial Sequence
  Primer
aagctccgagatctggacgag

SEQ ID NO: 18 - DNA, 21 bp, Artificial Sequence
  Primer
ctcgggaagcgcgccattgtg

SEQ ID NO: 19 - DNA, 24 bp, Artificial Sequence
  M13 Universal Primer
cgacgttgtaaaacgacggccagt

SEQ ID NO: 20 - DNA, 17 bp, Artificial Sequence
  M13 Reverse Primer
caggaaacagctatgac

SEQ ID NO: 21 - DNA, 20 bp, Artificial Sequence
  Primer CAP3
ggtcctatgtcaccggaatc

SEQ ID NO: 22 - DNA, 21 bp, Artificial Sequence
  Primer CAP4
ttccatgacgacccacactgc

SEQ ID NO: 23 - DNA, 21 bp, Artificial Sequence
  Primer 1RC2
caaggtacgccacagcgtatg

SEQ ID NO: 24 - DNA, 21 bp, Artificial Sequence
  Primer 2RC2
gtacgccacagcgtatgatgc

SEQ ID NO: 25 - DNA, 21 bp, Artificial Sequence
  Primer RTF1
agattccggtgacataggacc

SEQ ID NO: 26 - DNA, 21 bp, Artificial Sequence
  Primer RTR1
tggtctgggctctgtccctgc

SEQ ID NO: 27 - DNA, 21 bp, Artificial Sequence
  Sense Primer
caagagatggccacggctgct

SEQ ID NO: 28 - DNA, 21 bp, Artificial Sequence
  Anti-Sense Primer
tccttctgcatcctgtcggca

SEQ ID NO: 29 - DNA, 24 bp, Artificial Sequence
  Primer RTF2
atgagggggaatctggccctggtg

SEQ ID NO: 30 - DNA, 23 bp, Artificial Sequence
  Primer RTR2
catgttctccttgtcaaactcac

SEQ ID NO: 31 - DNA, 41 bp, Artificial Sequence
  Primer CL-L2-1F
gggaagcttcgatcaggatgagggggaatctggccctggtg

SEQ ID NO: 32 - DNA, 32 bp, Artificial Sequence
  Primer CL-L2-1R
gggctcgagcatgttctccttgtcaaactcac